(19) United States
(12) Patent Application Publication
(10) Pub. No.: US 2010/0047907 A1
(43) Pub. Date: Feb. 25, 2010

(54) METHODS OF TRANSPORTING EPITHELIAL CELL MONOLAYERS

(75) Inventors: Jibin Li, West Chester, PA (US); Paula S. Kardos, Wilmington, DE (US); Samantha Mackenzie Allen, Coatesville, PA (US); Ying Wang, Downingtown, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Absorption System Group LLC, Exton, PA (US)

(21) Appl. No.: 12/544,118

(22) Filed: Aug. 19, 2009

Related U.S. Application Data
(60) Provisional application No. 61/090,019, filed on Aug. 19, 2008.

(51) Int. Cl. C12N 5/08

(52) U.S. Cl. 435/371

(57) ABSTRACT

Described herein are tissue culture plates with permeable tissue culture plate inserts therein, which provides the tissue culture plates with apical chamber and a basolateral chambers, wherein cells are deposited on the permeable tissue culture inserts and essentially all of tissue culture medium has been removed from the apical chambers of the tissue culture plates and the basolateral chambers of the tissue culture plates contain a solidifiable form of cell culture medium. Also described are cells that can be deposited and grown on the described tissue culture inserts, methods for transporting a tissue culture plate with a permeable tissue culture plate insert therein on which cells are deposited. Also described is a kit for transporting the tissue culture plates described above, and corresponding methods of use.

### CROSS-REFERENCE TO RELATED APPLICATIONS

This application claims the benefit of U.S. Provisional Application No. 61/090,019, filed Aug. 19, 2008, which is incorporated by reference herein.

### TECHNICAL FIELD

The present invention relates to the field of cell culture.

### BACKGROUND

Epithelial cells play a vital role in vertebrate biology. These cells form epithelial barriers that are the guardians to the internal portions of the body. In this capacity, epithelial cells and the barriers they form serve two important functions: 1) segregating the internal and external cavities of the body and 2) providing a means for the body to selectively absorb and excrete particular substances. The functional role of epithelial barriers causes epithelial cells to be important in a variety of biological processes, such as chemical and nutrient absorption, waste excretion, and microbial infection.

Given the importance of epithelial cells to vertebrate biology, numerous model systems have been developed to study these cells and the epithelial barriers they compose. In some instances these model systems play a vital role in the development of new medicines or understanding various diseases. For example, polarized epithelial cells expressing proteins thought to be involved in the absorption of orally administered medications have been developed to study and characterize this process for existing and newly developed medicines. See, e.g., P. Balimane et al., AAPS J. 8:E1-13 (2006). Model systems of this sort are vital in drug development because they allow for the identification and characterization of candidate drugs and newly developed drugs. Therefore, epithelial membrane model systems can play a vital role in the development of new medical treatments.

While epithelial cell model systems are useful for drug discovery, working with the cells for this purpose can be difficult due to the highly uniform cell monolayers needed for this work. The parameters for experimental work of this sort range from choosing the proper cell type, to producing multiple uniform cell monolayers, to making sure cell monolayer integrity and polarization are sufficient to conduct the desired experiments. Furthermore, all of these parameters must be well-established to allow for repeated production of experimentally acceptable cell monolayers. These difficulties can make developing a desirable epithelial cell model system a daunting process, requiring months or years of work.

In some cases, a few of the difficulties of working with epithelial cell model systems have been minimized through the development of epithelial cell lines that can be used in drug discovery experiments. These established epithelial cell model systems can, in some cases, prevent end-users from having to develop their own model system, as these cells can often times be sent to the end-user frozen in dry ice. While the existence of these cell lines do minimize the overall efforts of an end-user in establishing an experimental model system, the cells still need to be maintained and cultured properly to produce the cell monolayers needed, a process that is not necessarily straightforward.

While the obstacles of growing, maintaining, and forming reproducible cell monolayers with cells being used in an epithelial cell model system could potentially be overcome by the commercial availability of the desired cell monolayers as a ready-to-use product, this alternative has the practical limitations related to shipping the cells, in a ready-to-use form, from the vendor to the end user. Unlike shipping frozen cells, as discussed above, live cells are usually shipped under conditions that at least approximate culture conditions for the cells, such as being covered with, or suspended in, growth medium. In the case of polarized cell monolayers, it is vital that the cells be shipped in such a way that cell monolayer integrity is not compromised because the integrity of epithelial cell monolayers is vital to the quality of this type of experimental system.

A method of shipping cell monolayers in a ready-to-use form would improve and streamline drug discovery/characterization processes that employ polarized cell monolayer systems.

### SUMMARY

Described herein is a cell culture comprising a tissue culture plate having a permeable tissue culture plate insert therein, where the permeable tissue culture plate insert provides the tissue culture plate with an apical chamber and a basolateral chamber and the permeable tissue culture insert has cells deposited thereon, where the apical chamber is essentially free of tissue culture medium and the basolateral chamber contains a solidifiable form of tissue culture medium.

The tissue culture plate inserts described provide a permeable growth support that can be inserted into a well of a tissue culture plate. The permeable insert itself does not take up the entire volume of the tissue culture well, rather it provides a means to partition the well into a bottom (basolateral) portion and top (apical) portion. Permeable tissue culture plate inserts serve to partition a tissue culture well by providing a growth substrate for a cell monolayer across the surface of the permeable support. Particularly in the case of polarized cells, once the monolayer is fully formed, it acts as a selective barrier between the apical and basolateral chambers of the tissue culture well. In some aspects, the permeable support of the tissue culture inserts described herein can be polycarbonate, polyester, polytetraflouroethylene, polystyrene, glass, cellulose, alumina, or polyethylene terephthalate, as well as other similar substances. In some aspects, the permeable support of the tissue culture inserts can be coated with one or more growth and/or differentiation substrates, such as collagen, fibronectin, laminin, vitronectin, D-lysine, and similar tissue culture substrates. The source of the growth and/or differentiation substrates may be from natural or synthetic sources.

Also described herein are cells that can be deposited and grown on the described tissue culture inserts. While any type of cell can be used, the most common cell types used are polarized cells, mammalian cells, epithelial cells, and even more preferably mammalian epithelial cells. Most preferably, the mammalian epithelial cells are polarized cells, such as Madin-Darby Canine Kidney (MDCK) cells, LLC PK1 porcine kidney cells, Caco-2 cells, CEBBe1 cells, HT-29 cells, T84 cells, and SK-CO15 cells, or derivative cells such as epithelial cells genetically engineered to express, or have reduced expression of, specific transporter proteins, such as human multidrug resistance protein 1 (MDR1), rodent mdr1 a or b, breast cancer resistance protein (BCRP), p-glycoprotein (PGP), multidrug resistance-associated protein 2 (MRP2), organic anion transporting polypeptide B1 (OATPB1) among others. In a preferred aspect, the derivative cells are polarized epithelial cells genetically engineered to have a reduced level of BCRP expression. In a more preferred aspect, the derivative cells are Caco-2 cells expressing a form of RNA that reduces the expression of BCRP. In one preferred aspect, the derivative cells are polarized epithelial cells genetically engineered to have a reduced level of PGP expression. In a more preferred aspect, the derivative cells are Caco-2 cells expressing a form of RNA that reduces the expression of PGP. In another preferred aspect, the derivative cells are polarized epithelial cells genetically engineered to have a reduced level of MRP2 expression. In a more preferred aspect, the derivative cells are Caco-2 cells expressing a form of RNA that reduces the expression of MRP2. In another preferred aspect, the derivative cells are polarized epithelial cells genetically engineered to express MDR1. In a more preferred aspect, the derivative cells are MDCK cells expressing MDR1.

Epithelial cells, polarized cells and polarized epithelial cells can be cultured on permeable tissue culture inserts and maintained in the presence of apical and basolateral tissue culture medium, especially in the form of a cell monolayer; however, these cell monolayers can also survive for extended periods of time in ambient conditions following the removal of the apical tissue culture medium. When the apical chamber is essentially free of tissue culture medium the basolateral tissue culture medium can be in a liquid, semisolid, or solid form. Accordingly, the basolateral medium can be supplemented with from about 0.05% to more than about 1% (w/v) of one or more solidifying agents, such as gelatin, collagen, xanthan gum, carob cassia, konjac gum, agarose, agar, pectin, guar gum, gum arabic, sodium alginate, carrageenan, irgacanth gum, hydroxyethyl methacrylaic, and the like. Accordingly, described herein are mammalian cells, epithelial cells, polarized cells, polarized epithelial cells and monolayers thereof maintained for extended periods of time, for example from about 3 to about 96 hours, in an ambient environment, outside of a tissue culture incubator, where the apical chamber of the tissue culture well is essentially free of medium and the basolateral chamber contains a solidifiable form of medium. Surprisingly, it has been observed that cells and cell monolayers maintained in this manner do not die following the extended incubation with essentially no medium in the apical chamber of the tissue culture well. For example, some cell cultures have been observed to be viable, as determined by cell monolayer integrity measurements, following about 1 to 3 days of incubation in an ambient environment with essentially no apical medium. Accordingly, disclosed herein are cells and cultures grown on permeable tissue culture plate inserts that are viable following periods of at least about 2 hours, about 5 hours, about 10 hours, about 20 hours, about 30 hours, about 40 hours, about 50 hours, about 60 hours, about 70 hours, about 80 hours, about 90 hours or more in an ambient environment, outside of a tissue culture incubator, where the apical chamber of the tissue culture well is essentially free of medium and the basolateral chamber contains a solidifiable form of medium.

Described herein are methods for transporting a cell culture plate grown on a permeable tissue culture plate insert placed in the well of a tissue culture plate such that the permeable tissue culture plate insert provides the tissue culture plate with an apical chamber and a basolateral chamber, wherein the apical chamber of the tissue culture plate is essentially free of tissue culture medium and the basolateral chamber of the tissue culture plate contains a solidifiable form of cell culture medium. In one aspect, the described cells are transported when they are shipped from one location to another location. In one aspect, the cells are transported for a period of at least 2 hours. In one aspect, the transported cells are mammalian cells. In one aspect, the transported cells are epithelial cells. In one aspect, the transported cells are polarized cells. In one aspect, the transported cells are in the form of a cell monolayer. Most preferably, the transported cells are polarized mammalian epithelial cells, such as MDCK cells, LLC PK1 porcine kidney cells, Caco-2 cells, CEBBe1 cells, HT-29 cells, T84 cells, and SK-CO15 cells, or derivative cells such as epithelial cell line engineered to express, or to have reduced expression of, specific transporter proteins, such as MDR1, rodent mdr1 a or b, BCRP, MRP2, or OATPB1, among others.

Also described herein are methods for receiving a cell culture plate grown on a permeable tissue culture plate insert placed in the well of a tissue culture plate such that the permeable tissue culture plate insert provides the tissue culture plate with an apical chamber and a basolateral chamber, wherein the apical chamber of the tissue culture plate is essentially free of tissue culture medium and the basolateral chamber of the tissue culture plate contains a solidifiable form of cell culture medium. In one aspect, the described cells are received when they are accepted or taken possession of following transport from one location to another location. In one aspect, the received cells are mammalian cells. In one aspect, the received cells are epithelial cells. In one aspect, the received cells are polarized cells. In one aspect, the received cells are in the form of a cell monolayer. Most preferably, the received cells are polarized mammalian epithelial cells, such as MDCK cells, LLC PK1 porcine kidney cells, Caco-2 cells, CEBBe1 cells, HT-29 cells, T84 cells, and SK-CO15 cells, or derivative cells such as epithelial cell line engineered to express, or to have reduced expression of, specific transporter proteins, such as MDR1, rodent mdr1 a or b, BCRP, MRP2, or OATPB 1, among others. In a preferred aspect, the derivative cells are polarized epithelial cells genetically engineered to have a reduced level of BCRP expression. In a more preferred aspect, the derivative cells are Caco-2 cells expressing a form of RNA that reduces the expression of BCRP. In one preferred aspect, the derivative cells are polarized epithelial cells genetically engineered to have a reduced level of PGP expression. In a more preferred aspect, the derivative cells are Caco-2 cells expressing a form of RNA that reduces the expression of PGP. In another preferred aspect, the derivative cells are polarized epithelial cells genetically engineered to have a reduced level of MRP2 expression. In a more preferred aspect, the derivative cells are Caco-2 cells expressing a form of RNA that reduces the expression of MRP2. In another preferred aspect, the derivative cells are polarized epithelial cells genetically engineered to express MDR1. In a more preferred aspect, the derivative cells are MDCK cells expressing MDR1.

Also included herein are methods for feeding or replenishing the medium of cells and cell monolayers cultured on permeable tissue culture well inserts, where the tissue culture plates have an apical chamber that is essentially free of tissue culture medium and a basolateral chamber that contains a solidifiable form of cell culture medium. For example, medium can be added to the apical chamber, and the solidified medium can be converted to a liquid form and aspirated or poured from the plate, or it can be extracted while still in solid form, or the porous tissue culture insert can be removed from the well having the solidified medium and added to a different well having liquid growth medium, and, in some instances, liquid medium or biological buffer can be added to the basolateral chamber of the well having solidified basolateral medium to facilitate removal of the porous tissue culture insert or the solidified tissue culture medium. Also described herein are methods for feeding or replenishing the medium of cells and cell monolayers cultured on permeable tissue culture well inserts that include supplementing the culture medium with a chemical agent that facilitates cell differentiation, such as a salt or ester of butyric acid. For example, a salt of butyric acid can be sodium butyrate, potassium butyrate, sodium butanoate, and the like. See e.g., Olmo, N. et al., In vitro models for the study of the effect of butyrate on human colon adenocarcinoma cells. Toxicology In Vitro, 21(2):262-270 (2007).

Described herein is a kit for transporting cells grown in a tissue culture plate having a permeable tissue culture plate insert therein, where the permeable tissue culture plate insert provides the tissue culture plate with an apical chamber and a basolateral chamber, and the cells are deposited on the permeable tissue culture insert, where the apical chamber of the tissue culture plate is essentially free of tissue culture medium and the basolateral chamber of the tissue culture plate contains a solidifiable form of tissue culture medium. In one embodiment, the described kit contains liquid tissue culture medium that can be used to feed the cells included in the kit. The described kit can also have instruction that allow an end user to make use of the kit and its contents. Also described are methods for using the kit to transport cells grown in a tissue culture plate having a permeable tissue culture plate insert therein, where the permeable tissue culture plate insert provides the tissue culture plate with an apical chamber and a basolateral chamber, and the cells are deposited on the permeable tissue culture insert, where the apical chamber of the tissue culture plate is essentially free of tissue culture medium and the basolateral chamber of the tissue culture plate contains a solidifiable form of tissue culture medium.

### BRIEF DESCRIPTION OF THE DRAWINGS

FIG. 1 provides a schematic representation of a single well of a tissue culture plate (6) having a permeable tissue culture plate insert (7) therein, where a cell monolayer (2) is present on the permeable growth support (1). Cell culture medium (5) is depicted in the basolateral chamber of the plate (3), while the apical chamber of the plate (4) does not contain cell culture medium.

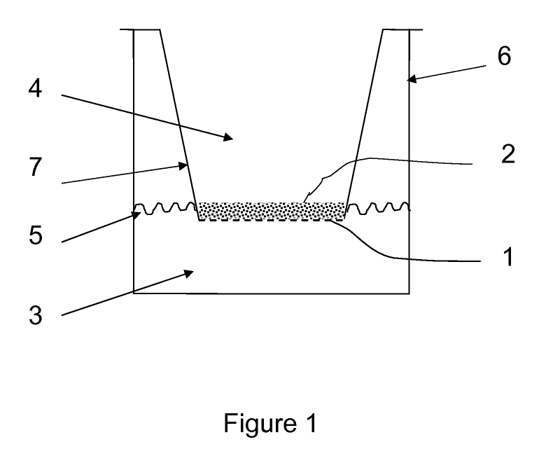

FIG. 2 provides a side view (A) and top view (B) of a schematic representation of a 12-well tissue culture plate.

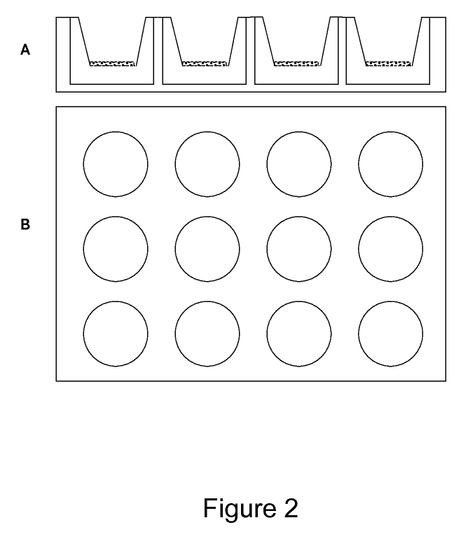

### DETAILED DESCRIPTION OF ILLUSTRATIVE EMBODIMENTS

Various terms relating to the methods and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

The following abbreviations are used throughout the specification. Papp: permeability coefficient; TEER: transepithelial electrical resistance; MDCK: Madin-Darby canine kidney; MDR1-MDCK: MDCK cell line transfected with the human MDR1 gene; MDR1: multidrug resistance protein 1; BCRP: breast cancer resistance protein; MRP2: multidrug resistance-associated protein 2; OATPB 1: organic anion transporting polypeptide B 1.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “ambient conditions” or “ambient environment” as used herein refer to the general, common atmospheric and weather conditions indoors or outdoors, as distinguished from the highly regulated atmospheric conditions of a tissue culture incubator. For example, as used herein these terms can apply equally to a laboratory, the cargo hold of a truck or plane, or the interior of a parcel.

The term “essentially all” as used herein when referring to the removal of tissue culture medium means removal of almost all medium with the understanding that some residual amount of medium will remain due to adhesive forces between the medium and tissue culture apparatus.

The term “essentially free of tissue culture medium” means free of almost all tissue culture medium, with the understanding that some residual amount of medium will remain due to adhesive forces between the medium and tissue culture apparatus and cell surface.

The term “derivative cell” means a cell type that was derived from another cell type by techniques such as subcloning, transfection of plasmid nucleic acid, transformation, transduction, mutagenesis, or other genetic engineering technique. For example, the MDR-MDCK cell line was produced by transfecting the MDCK cell line with the human MDR1 gene; therefore, the MDR-MDCK cell line is an MDCK derivative cell line.

The term “solidifiable” as used herein when referring to tissue culture medium means capable of being a solid at ambient temperature, but may be found in an alternative state, such as a liquid state.

The term “transport,” and words derived therefrom, as used herein when referring to moving cells can refer to the entire transport process or any particular aspect of transport, such as packaging for transport, shipping, or the act of moving from one location to another.

The term “receive,” and words derived therefrom, as used herein when referring to moving cells means to accept or take possession of something that has been transported.

It is to be understood that this invention is not limited to particular methods, reagents, compounds, compositions or biological systems, which can, of course, vary. It is also to be understood that the terminology used herein is for the purpose of describing particular aspects only, and is not intended to be limiting.

Epithelial cells are widely used to study a variety of biological processes. The variability of the cell types that compose mammalian epithelium provides a range of cell types that are well suited for studies related to molecular cell biology, microbial pathogenesis, and pharmacology, to name a few. Epithelial cell lines can be classified as polarized or nonpolarized. While both cell types are useful in the study of epithelial biology, cells often lend themselves to very different scientific studies based on whether or not they polarize.

Polarized epithelial cells have a number of characteristics that distinguish them from nonpolarized epithelial cells. One distinguishing feature is the formation of tight junctions that segregate the plasma membrane into apical and basolateral portions. The apical portion of the cell is the exposed, or top, portion of the cell when oriented in a cell monolayer grown on a tissue culture plate; however, in the context of a polarized cell in an epithelial cell sheet in the body, the apical surface would be exposed to the lumen lined by the epithelium. The basolateral surface of the cell is actually composed of two portions of the cell: the bottom, or basal, portion and the side, or lateral, portions. In the context of a cell grown on a tissue culture plate, the basolateral membrane of the cell would be the portion of the cell contacting the tissue culture plate and the lateral portion of the cell situated below the tight junctions. In the context of a polarized cell in an epithelial cell sheet in the body, the basolateral surface of the cell would be exposed to the internal portion of the body lined by the epithelium.

In some instances cellular proteins are expressed in a polarized manner. For example, certain ion transporters, such as the epithelium sodium channel, cellular receptor proteins, such as the polymeric immunoglobulin receptor, are preferentially expressed on either the apical or basolateral surface of polarized epithelial cells. C. Planès and G. H. Caughey, Curr. Top. Dev. Biol.78:23-46 (2007); W. Song, et al., Proc. Natl. Acad. Sci. U. S. A. January 4;91(1):163-6 (1994). In addition, some proteins expressed in polarized manner are used to transport extracellular proteins from one side of an epithelial cell to another. W. Song, et al.

Often studying the biology of proteins expressed in a polarized manner requires the ability to access and, in some cases, modify the culture medium that is in contact with the apical or basolateral surfaces of the cell. Of course, standard tissue culture devices do not allow for this sort of manipulation; therefore, specialized cell culture devices have been developed to allow for the study polarized processes of epithelial cells. The primary device used for this purpose is a permeable tissue culture plate insert, such as a Transwell® (Corning, Inc., Lowell, Mass.). These devices provide a permeable growth support that can be inserted into a well of a tissue culture plate. The permeable insert itself does not take up the entire volume of the tissue culture well, rather it provides a means to partition the well into a basolateral portion and apical portion. The partition is actually made by culturing a polarized cell monolayer across the surface of the permeable growth support. Once the cell membrane covers the entire permeable growth support and becomes polarized, it will function as a selective barrier to separate the apical and basolateral chambers of the tissue culture well.

Polarized cell monolayers grown on permeable tissue culture inserts provide a basic experimental system in which one can examine polarized cell processes, such as polarized transport or transcytosis. Typically these processes are studied by placing a substance of interest in either the apical or basolateral chamber of a tissue culture well having an insert with a polarized cell monolayer and assessing whether or not the substance is transported to the opposite chamber. In addition to determining how a transport protein functions to absorb or excrete proteins in a polarized manner, one can also use polarized cell systems to screen for inhibitors of such transport proteins. Experiments of this sort are important in the context of minimizing the interference of efflux transporters, such as PGP, in the process of drug delivery.

One thing that is common to all of the various types of polarized epithelial cell studies one can perform is, of course, a polarized epithelial cell monolayer that provides apical and basolateral chambers of a tissue culture well. Unfortunately, producing these monolayers is not always straight-forward and often requires specialized knowledge of how to culture and maintain the cells of interest. For example, one must know the proper type of permeable substrate to which the cells will adhere and form a polarized monolayer; understand the growth characteristics of the cells to assure that cell monolayers are not over confluent when experiments are conducted; be familiar with how to assess cell monolayer integrity to assure that cell monolayers are acting as a selective barrier between the partitioned compartments of the well; and, where statistical analysis requires a large number of simultaneous experiments, be able to produce a large number of substantially similar polarized cell monolayers. This multitude of parameters highlights only some of the confounding variables that make experiments involving polarized epithelia cells difficult to execute properly.

In today's scientific world, the problem of using experimental reagents that are difficult to produce or handle is often overcome by using a commercially available form of the reagent. A wonderful example of this practice is provided in the context of polyacrylamide gel electrophoresis. Polyacrylamide gels provide a powerful tool by which one can assess proteins; therefore, these gels are a common research tool for the molecular biologist. These gels, however, can be difficult and time consuming to make in the lab, which hinders efficient production of experimental results. This problem was alleviated, to a large degree, when commercial vendors created electrophoresis systems in which pre-made, disposable polyacrylamide gels could be used. Now many research labs have forgone the inefficient and frustrating process of making polyacrylamide gels and instead order the gels from commercial vendors.

The ease and efficiency with which many researchers conduct polarized cell experiments would be increased if polarized cell monolayers were available in a ready-to-use form. Unfortunately, it is not as easy for commercial vendors to provide polarized epithelial cell monolayers as it is to provide polyacrylamide gels or analogous reagents. A major obstacle to making polarized cell monolayers commercially available is that live cell monolayers would need to be shipped to consumers. Shipping the cells as live, intact, polarized cell monolayers presents a variety of problems because the cells are maintained and cultured in liquid medium, in a highly controlled environment unlike that of the ambient environment.

Described herein is a cell culture grown in a tissue culture plate having a permeable tissue culture plate insert therein, where the permeable tissue culture plate insert provides the tissue culture plate with an apical chamber and a basolateral chamber and the permeable tissue culture insert has cells deposited thereon, where the apical chamber is essentially free of tissue culture medium and the basolateral chamber contains a solidifiable form of tissue culture medium.

### Tissue Culture Inserts

Studying the biology of proteins expressed in a polarized manner often requires the ability to access and, in some cases, modify the culture medium that is in contact with the apical or basolateral surfaces of the cell. The primary device used for this purpose is a permeable tissue culture plate insert, such as a Transwell®. These devices provide a permeable growth support that can be inserted into a well of a tissue culture plate. The permeable insert itself does not take up the entire volume of the tissue culture well, rather it provides a means to partition the well into a basolateral portion and apical portion. The well is actually partitioned by culturing a polarized cell monolayer across the surface of the permeable growth support. Once the cell membrane covers the entire permeable growth support and becomes polarized, it will function as a selective barrier to separate the apical and basolateral chambers of the tissue culture well.

In some aspects, the permeable support of the tissue culture inserts described herein can be polycarbonate, polyester, polytetrafluoroethylene, polystyrene, glass, cellulose, alumina, or polyethylene terephthalate, as well as other similar substances. Accordingly, in one aspect the tissue culture plate insert described herein is a tissue culture insert having a permeable support made of polycarbonate. In one aspect the tissue culture plate insert having a permeable support made of polycarbonate is a Transwell® insert. In one aspect the tissue culture plate insert having a permeable support made of polycarbonate is a Millicell™ insert. In one aspect, the tissue culture plate insert described herein is a tissue culture insert having a permeable support made of polyester. In one aspect the tissue culture plate insert having a permeable support made of polyester is a Transwell® insert. In one aspect, the tissue culture plate insert described herein is a tissue culture insert having a permeable support made of polytetraflouroethylene. In one aspect the tissue culture plate insert having a permeable support made of polytetraflouroethylene is a Transwell® insert. In one aspect the tissue culture plate insert having a permeable support made of polytetraflouroethylene is a Millicell™ insert. In one aspect, the tissue culture plate insert described herein is a tissue culture insert having a permeable support made of polystyrene. In one aspect, the tissue culture plate insert described herein is a tissue culture insert having a permeable support made of glass. In one aspect, the tissue culture plate insert described herein is a tissue culture insert having a permeable support made of cellulose. In one aspect the tissue culture plate insert having a permeable support made of cellulose is a Millicell™ insert. In one aspect, the tissue culture plate insert described herein is a tissue culture insert having a permeable support made of an alumina membrane. In one aspect the tissue culture plate insert having a permeable support made of an alumina membrane is an Anopore® membrane insert. In one aspect, the tissue culture plate insert described herein is a tissue culture insert having a permeable support made of a polyethylene terephthalate membrane.

In some aspects the permeable support of the tissue culture inserts can be coated with one or more growth and/or differentiation substrates, such as collagen, fibronectin, laminin, vitronectin, D-lysine, and similar tissue culture substrates. Accordingly, in one aspect, permeable support of the tissue culture inserts can be coated with a collagen substrate. In one aspect, permeable support of the tissue culture inserts can be coated with a collagen I substrate. In another aspect, permeable support of the tissue culture inserts can be coated with a collagen IV substrate. In one aspect, permeable support of the tissue culture inserts can be coated with a fibronectin substrate. In one aspect, permeable support of the tissue culture inserts can be coated with a laminin substrate. In one aspect, permeable support of the tissue culture inserts can be coated with a vitronectin substrate. In one aspect, permeable support of the tissue culture inserts can be coated with a D-lysine substrate. In a preferred aspect, the permeable support is a provided by a Transwell® insert coated with rat tail collagen. Other such growth and differentiation substrates are known in the art and are considered within the scope of this disclosure. Furthermore, such substrates can be used in any combination.

The source of the growth and/or differentiation substrates may be from natural or synthetic sources. In some aspects, the coating may be derived from an animal, such as primates, avians, rodents, and the like. Accordingly, in one aspect the growth and/or differentiation substrate is derived from a human source, such as blood, a cell, a tissue, or a gene. In one aspect the growth and/or differentiation substrate is derived from a mouse source, such as blood, a cell, a tissue, or a gene. In another aspect the growth and/or differentiation substrate is derived from a rat source, such as blood, a cell, a tissue, or a gene. In one aspect the growth and/or differentiation substrate is derived from a cow source, such as blood, a cell, a tissue, or a gene. In one aspect the growth and/or differentiation substrate is derived from a chicken source, such as blood, a cell, a tissue, or a gene. In one aspect the growth and/or differentiation substrate is derived from a horse, such as blood, a cell, a tissue, or a gene. In some aspects, the substrate may be synthesized either chemically or biologically. For example, in some aspects the substrate can be harvested from genetically engineered bacteria that express the substrate. In another aspect, the substrate may be synthesized chemically in a laboratory or factory setting. In some aspects, the substrate may be derived from a plant.

### Cells

Described herein are cells grown in a tissue culture plate having a permeable tissue culture plate insert therein, where the permeable tissue culture plate insert provides the tissue culture plate with an apical chamber and a basolateral chamber and the permeable tissue culture insert has cells deposited thereon, where the apical chamber is essentially free of tissue culture medium and the basolateral chamber contains a solidifiable form of tissue culture medium. While any type of cell able to form a cell monolayer on a tissue culture insert can be used as described herein, the most common cell types used are epithelial cells, mammalian cells, mammalian epithelial cells, polarized cells, and, most preferably, polarized mammalian epithelial cells. The cells described herein can be cultured as a collection of single cells, a culture in the form of a cell monolayer, or a mixture of both. Mammalian epithelial cells can be derived from a variety of sources, such as humans, apes, cattle, rodents, canines, felines, etc. Furthermore, mammalian epithelial cells can be derived from a variety of organs, such as kidney, colon, intestine, and lung. Accordingly, the cells could be MDCK cells, LLC PK1 porcine kidney cells, Caco-2 cells, CEBBe1 cells, HT-29 cells, T84 cells, and SK-CO15 cells, or derivative cell lines such as epithelial cell lines engineered to express, or to have reduced expression of, specific transporter proteins, such as MDR1, rodent mdr1 a or b, BCRP, MRP2, or OATPB1, among others. In a preferred aspect, the derivative cells are polarized epithelial cells genetically engineered to have a reduced level of BCRP expression. In a more preferred aspect, the derivative cells are Caco-2 cells expressing a form of RNA that reduces the expression of BCRP. In one preferred aspect, the derivative cells are polarized epithelial cells genetically engineered to have a reduced level of PGP expression. In a more preferred aspect, the derivative cells are Caco-2 cells expressing a form of RNA that reduces the expression of PGP. In another preferred aspect, the derivative cells are polarized epithelial cells genetically engineered to have a reduced level of MRP2 expression. In a more preferred aspect, the derivative cells are Caco-2 cells expressing a form of RNA that reduces the expression of MRP2. In another preferred aspect, the derivative cells are polarized epithelial cells genetically engineered to express MDR1. In a more preferred aspect, the derivative cells are MDCK cells expressing MDR1. There are other mammalian epithelia cell lines that are well known in the art, which are considered within the scope of this disclosure. Furthermore, cells derived from those cell lines described herein, such as genetically modified variants are also within the scope of this disclosure.

A hallmark of cultured polarized cells is their ability to form polarized cell monolayers having high transepithelial electrical resistance (TEER), relative to non-polarized cells. The degree of TEER can vary widely among polarized cell monolayers, depending on the cell type and the extent to which the cells form intercellular connections, such as tight junctions. M. Laukoetter, et al., J. Exp. Med. 204(13):3067-76 (2007). In some aspects, the cells and cell monolayers described herein have the ability to form cell monolayers having high TEER. In some aspects, the polarized epithelial cell monolayers have a TEER value of at least 50 Ω∩cm². In some aspects, the polarized epithelial cell monolayers have a TEER value of at least 100 Ω·cm². In some aspects, the polarized epithelial cell monolayers have a TEER value of at least 150 Ω·cm². In some aspects, the polarized epithelial cell monolayers have a TEER value of at least 200 Ω·cm². In some aspects, the polarized epithelial cell monolayers have a TEER value of at least 250 Ω·cm². In some aspects, the polarized epithelial cell monolayers have a TEER value of at least 300 Ω·cm². In some aspects, the polarized epithelial cell monolayers have a TEER value of at least 350 Ω·cm ². In some aspects, the polarized epithelial cell monolayers have a TEER value of at least 400 Ω·cm². In some aspects, the polarized epithelial cell monolayers have a TEER value of at least 450 Ω·cm². In some aspects, the polarized epithelial cell monolayers have a TEER value of at least 500 Ω·cm². Those of skill in the art will understand that TEER values for polarized monolayers can reach much higher than those values set forth above; therefore these values should not be viewed as limits on the TEER values of polarized epithelia monolayers. For example, in some applications the cell monolayers described herein have TEER values above 500 Ω·cm² or even above 1000, 1100, 1200, 1300, 1400, 1500 Ω·cm² or more. In some aspects cell monolayers having high TEER values are composed of epithelial cells. In some aspects cell monolayers having high TEER values are composed of mammalian cells. In some aspects cell monolayers having high TEER values are composed of mammalian epithelial cells. In some aspects cell monolayers having high TEER values are composed of polarized mammalian epithelial cells.

In addition to TEER, the integrity of polarized epithelial cell monolayers can be assessed by examining the ability of molecules to passively diffuse across the cell monolayer. Lucifer yellow is a fluorescent molecule that can be used to determine the integrity of a polarized epithelial monolayer. In some aspects, the cells and cell monolayers described herein have the ability to form cell monolayers that inhibit the passive diffusion of lucifer yellow. In some aspects, polarized cell monolayers will permit diffusion of lucifer yellow at a rate less than about 0.05×10⁻⁶ cm/s. In some aspects, polarized cell monolayers will permit diffusion of lucifer yellow at a rate less than about 0.1×10⁻⁶ cm/s. In some aspects, polarized cell monolayers will permit diffusion of lucifer yellow at a rate less than about 0.2×10⁻⁶ cm/s. In some aspects, polarized cell monolayers will permit diffusion of lucifer yellow at a rate less than about 0.3×10⁻⁶ cm/s. In some aspects, polarized cell monolayers will permit diffusion of lucifer yellow at a rate less than about 0.4×10⁻⁶ cm/s. In some aspects, polarized cell monolayers will permit diffusion of lucifer yellow at a rate less than about 0.5×10⁻⁶ cm/s. In some aspects, polarized cell monolayers will permit diffusion of lucifer yellow at a rate less than about 0.6×10⁻⁶ cm/s. In some aspects, polarized cell monolayers will permit diffusion of lucifer yellow at a rate less than about 0.7×10⁻⁶ cm/s. In some aspects, polarized cell monolayers will permit diffusion of lucifer yellow at a rate less than about 0.8×10-6 cm/s. In some aspects, polarized cell monolayers will permit diffusion of lucifer yellow at a rate less than about 0.9×10-6 cm/s. In some aspects, polarized cell monolayers will permit diffusion of lucifer yellow at a rate less than about 1×10⁻⁶ cm/s. In some aspects cell monolayers that inhibit the passive diffusion of lucifer yellow are composed of epithelial cells. In some aspects cell monolayers that inhibit the passive diffusion of lucifer yellow are composed of mammalian cells. In some aspects cell monolayers that inhibit the passive diffusion of lucifer yellow are composed of mammalian epithelial cells. In some aspects cell monolayers that inhibit the passive diffusion of lucifer yellow are composed of polarized mammalian epithelial cells.

### Maintenance of Cultured Polarized Epithelial Cells

Epithelial cell monolayers are often cultured on permeable tissue culture plate inserts to allow for the study of various aspects of polarized cell biology or epithelial biology. Typically, the cells are cultured and maintained in the presence of apical and basolateral tissue culture medium. In addition, the cells are usually grown and maintained in a controlled environment, such as a tissue culture incubator, which maintains desirable cell culture conditions, such as 37° C., 5% CO₂, and 95% relative humidity. Because cells are cultured under specific conditions, which are vastly different from the typical ambient environment of a laboratory, tissue culture medium is formulated to maintain cells in the conditions of a tissue culture incubator. For these reasons, among others, it was thought that cells exposed to ambient environmental conditions would not survive for more that a short period of time.

Surprisingly, it has been determined that cultured epithelial cell monolayers can not only survive for extended periods of time in ambient conditions but can do so in the absence of apical tissue culture medium. As described above, the cells can be MDCK cells, Caco-2 cells, CEBBe1 cells, HT-29 cells, T-84 cells, and SK-CO15 cells, or derivative cells, as well as other cell types known in the art. In some aspects, the cell culture can be attached to a permeable cell culture insert wherein the apical chamber is essentially free of tissue culture medium. In some aspects, the cell culture is attached to a permeable cell culture insert where essentially all of the apical tissue culture medium is removed. In some aspects, the basolateral tissue culture medium can be in a liquid form. In another aspect, the basolateral tissue culture medium can be in a solid form. Accordingly, the basolateral medium can be supplemented with one or more solidifying agents, such as gelatin, collagen, xanthan gum, carob cassia, konjac gum, agarose, agar, pectin, guar gum, gum arabic, sodium alginate, carrageenan, irgacanth gum, hydroxyethyl methacrylaic, and the like. Aspects of the basolateral tissue culture medium include those in which the medium can be supplemented with about 0.05%, about 0.1%, about 0.2%, 0.3%, about 0.4%, about 0.5%, about 0.6%, about 0.7%, about 0.8%, about 0.9%, about 1.0% (w/v), or more of a solidifying agent. In a preferred aspect, the basolateral tissue culture medium is supplemented with from about 0.5% to about 1.0% (w/v) agarose. In a more preferred aspect, basolateral tissue culture medium is supplemented with about 0.75% (w/v) agarose. In some aspects, the basolateral tissue culture medium is still in liquid form even after the solidifying agent is added. In another aspect, the basolateral tissue culture medium is solidified after the solidifying agent is added. In another aspect, the basolateral tissue culture medium is heated to maintain it as a liquid following addition the solidifying agent. In one aspect, the tissue culture medium is cooled to convert it to solid form following addition the solidifying agent.

Described herein are cell cultures grown on porous tissue culture inserts that can survive for an extended period of time in an ambient environment where the apical chamber of a tissue culture well having a porous insert is essentially free of tissue culture medium and a solidifiable form of tissue culture medium is in the basolateral chamber the tissue culture well. While any type of cell able to form a cell monolayer on a tissue culture insert can be used as described, the most common cell types used are epithelial cells, mammalian cells, mammalian epithelial cells, polarized cells, and, most preferably, polarized mammalian epithelial cells. Accordingly, in some aspects, cell cultures on a porous tissue culture insert are viable after at least about 5 hours in an ambient environment where the apical chamber is essentially free of tissue culture medium and a solidifiable form of tissue culture medium is in the basolateral chamber. In some aspects, cell cultures on a porous tissue culture insert are viable after at least about 12 hours in an ambient environment where the apical chamber is essentially free of tissue culture medium and a solidifiable form of tissue culture medium is in the basolateral chamber. In some aspects, cell cultures on a porous tissue culture insert are viable after at least about 18 hours in an ambient environment where the apical chamber is essentially free of tissue culture medium and a solidifiable form of tissue culture medium is in the basolateral chamber. In some aspects, cell cultures on a porous tissue culture insert are viable after at least about 24 hours in an ambient environment where the apical chamber is essentially free of tissue culture medium and a solidifiable form of tissue culture medium is in the basolateral chamber. In some aspects, cell cultures on a porous tissue culture insert are viable after at least about 36 hours in an ambient environment where the apical chamber is essentially free of tissue culture medium and a solidifiable form of tissue culture medium is in the basolateral chamber. In some aspects, cell cultures on a porous tissue culture insert are viable after at least about 48 hours in an ambient environment where the apical chamber is essentially free of tissue culture medium and a solidifiable form of tissue culture medium is in the basolateral chamber. In some aspects, cell cultures on a porous tissue culture insert are viable after at least about 60 hours in an ambient environment where the apical chamber is essentially free of tissue culture medium and a solidifiable form of tissue culture medium is in the basolateral chamber. In some aspects, cell cultures on a porous tissue culture insert are viable after at least about 72 hours in an ambient environment where the apical chamber is essentially free of tissue culture medium and a solidifiable form of tissue culture medium is in the basolateral chamber. In some aspects, cell cultures on a porous tissue culture insert are viable after at least about 84 hours in an ambient environment where the apical chamber is essentially free of tissue culture medium and a solidifiable form of tissue culture medium is in the basolateral chamber. In some aspects, cell cultures on a porous tissue culture insert are viable after at least about 96 hours, or more, in an ambient environment where the apical chamber is essentially free of tissue culture medium and a solidifiable form of tissue culture medium is in the basolateral chamber.

Some of the cell types described herein will form cell monolayers that are not compromised following extended periods of time without apical tissue culture medium, as described previously. Following a period of incubation under normal growth conditions (i.e. incubation in a tissue culture incubator where the there is liquid, non-solidifiable tissue culture medium in both the apical and basolateral chambers of the tissue culture plate insert), the TEER and passive diffusion of lucifer yellow can be as good or better than that of cell monolayers continually maintained under normal growth conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 100 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 150 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 200 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 250 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 300 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 350 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 400 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 450 Ω·cm² about 8 to about 48 hours after incubation under normal grown conditions. In some aspects, a cell monolayer on a porous tissue culture insert in an ambient environment for at least 5 hours, where the apical chamber is essentially free of tissue culture medium and a solidifiable form of tissue culture medium is in the basolateral chamber, has a TEER of at least 500 Ω·cm² about 8 to about 48 hours after incubation under normal grown conditions. In some aspects, the TEER of the cell monolayers is greater than 500 Ω·cm² and may even be greater than 1000, 1100, 1200, 1300, 1400, 1500 Ω·cm² or more, about 8 to about 48 hours after incubation under normal grown conditions. Similarly, these cell monolayers will also inhibit the passive diffusion of lucifer yellow, as described herein. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.05×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.1×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.2×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.3×10⁻⁶ cm/s about 8 to about 48 hours after incubation under normal grown conditions. In some aspects, the passive diffusion of lucifer yellow across cell monolayers will on a porous tissue culture insert in an ambient environment for at least 5 hours, where the apical chamber is essentially free of tissue culture medium and a solidifiable form of tissue culture medium is in the basolateral chamber, be less than about 0.4×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.5×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.6×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.7×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.8×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 0.9×10⁻⁶ cm/s about 8 to about 48 hours after incubation under normal grown conditions. In some aspects, the passive diffusion of lucifer yellow across cell monolayers on a porous tissue culture insert in an ambient environment for at least 5 hours, where the apical chamber is essentially free of tissue culture medium and a solidifiable form of tissue culture medium is in the basolateral chamber, will be less than about 1.0×10⁻⁶ cm/s about 8 to about 48 hours after incubation under normal grown conditions. As described previously, exposure to an ambient environment can include the process of transporting or shipping cell cultures outside of a tissue culture incubator.

In order to effectively replenish the basolateral medium for cultured cell monolayers having solidified medium in the basolateral chamber, the solidified basolateral medium must be removed. For example, in one aspect, the solidified medium can be converted to a liquid form and aspirated or poured from the plate. In another aspect, the solidified medium can be extracted while still in solid form. In one aspect, the solidified basolateral medium is not removed, but rather the porous tissue culture insert is removed from the well having the solidified medium and added to a different well having liquid tissue culture medium. In some instances, liquid medium or biological buffer can be added to the basolateral chamber of the well having solidified basolateral medium to facilitate removal of the porous tissue culture insert from the solidified tissue culture medium. Those of skill in the art will realize that the basolateral medium can be removed in a number of ways without damaging the cell monolayer on the porous insert. Following removal of the solidified basolateral medium, liquid tissue culture medium can be added to the basolateral chamber. In each of the aspects described in this paragraph, the apical medium can be replaced by simply adding it to the apical chamber of the tissue culture insert.

In some aspects the described methods for feeding or replenishing the medium of cells and cell monolayers cultured on permeable tissue culture well inserts include supplementing the medium with a chemical agent, such as a salt of butyric acid (e.g. sodium butyrate). For example, medium supplemented with sodium butyrate can be used to incubate cell monolayers removed from tissue culture plates containing solidified growth medium. Alternatively, sodium butyrate can be used to supplement medium used prior to the addition of a solidifiable form of cell culture medium. In some embodiments, a chemical agent, such as sodium butyrate, can be used to supplement the solidifiable forms of cell culture medium described herein. The amount of sodium butyrate used to supplement the media described herein can vary, based on several factors such as culture conditions, transport conditions, cell type, and the type of growth medium used. In some embodiments cell culture medium can be supplemented to contain about 4 mM sodium butyrate. However, in other embodiments the cell culture medium can contain concentrations greater or less than about 4 mM sodium butyrate. Accordingly, in some embodiments medium can contain about 1 mM, about 2 mM, about 3 mM, about 4 mM, about 5 mM, about 6 mM, about 7 mM, about 8 mM. about 9 mM, about 10 mM, about 11 mM, about 12 mM, about 13 mM, about 14 mM, about 15 mM, about 16 mM, about 17 mM, about 18 mM. about 19 mM, or about 20 mM sodium butyrate.

### Cell Transport

The ability to maintain cell cultures in ambient conditions by providing nothing more than solidified tissue culture medium in the basolateral chamber of a tissue culture plate allows the cells to be used in new ways, not possible for cells that cannot tolerate such conditions. One process that the cells can undergo is transport, for example, shipping over a substantial distance or transporting the described cell cultures for a period of at least 2 hours. Because the cells can tolerate ambient conditions for extended periods of time, it is possible for them to be shipped as live cell cultures. Accordingly, described herein are methods for transporting a cell culture on a tissue culture plate having a permeable tissue culture plate insert therein, said permeable tissue culture plate insert providing the tissue culture plate with an apical chamber and a basolateral chamber, wherein cells are deposited on the permeable tissue culture insert, wherein the apical chamber of the tissue culture plate is essentially free of tissue culture medium and the basolateral chamber of the tissue culture plate contains a solidifiable form of cell culture media. In one aspect, the described cells are used for shipping when they are transported from one location to another location. While any type of cell able to form a cell monolayer on a tissue culture insert can be transported as described, the most common cell types used are epithelial cells, mammalian cells, mammalian epithelial cells, polarized cells, and, most preferably, polarized mammalian epithelial cells. Accordingly, the cells could be MDCK cells, LLC PK1 porcine kidney cells, Caco-2 cells, CEBBe1 cells, HT-29 cells, T84 cells, and SK-CO15 cells, or derivative cell lines such as epithelial cell lines engineered to express, or to have reduced expression of, specific transporter proteins, such as MDR1, rodent mdr1 a or b, BCRP, MRP2, or OATPB1, among others. In a preferred aspect, the derivative cells are polarized epithelial cells genetically engineered to have a reduced level of BCRP expression. In a more preferred aspect, the derivative cells are Caco-2 cells expressing a form of RNA that reduces the expression of BCRP. In one preferred aspect, the derivative cells are polarized epithelial cells genetically engineered to have a reduced level of PGP expression. In a more preferred aspect, the derivative cells are Caco-2 cells expressing a form of RNA that reduces the expression of PGP. In another preferred aspect, the derivative cells are polarized epithelial cells genetically engineered to have a reduced level of MRP2 expression. In a more preferred aspect, the derivative cells are Caco-2 cells expressing a form of RNA that reduces the expression of MRP2. In another preferred aspect, the derivative cells are polarized epithelial cells genetically engineered to express MDR1. In a more preferred aspect, the derivative cells are MDCK cells expressing MDR1. As will now be apparent to those of skill in the art, any number of cell lines that are found to persist using the methods described herein will also lend themselves to the transport methods described herein, such aspects are considered to be within the scope of the described methods.

Also described herein are methods for receiving a cell culture plate grown on a permeable tissue culture plate insert placed in the well of a tissue culture plate such that the permeable tissue culture plate insert provides the tissue culture plate with an apical chamber and a basolateral chamber, wherein the apical chamber of the tissue culture plate is essentially free of tissue culture medium and the basolateral chamber of the tissue culture plate contains a solidifiable form of cell culture medium. In one aspect, the described cells are received when they are accepted or taken possession of following transport from one location to another location. In one aspect, the received cells are mammalian cells. In one aspect, the received cells are epithelial cells. In one aspect, the received cells are polarized cells. In one aspect, the received cells are in the form of a cell monolayer. Most preferably, the received cells are polarized mammalian epithelial cells, such as MDCK cells, LLC PK1 porcine kidney cells, Caco-2 cells, CEBBe1 cells, HT-29 cells, T84 cells, and SK-C015 cells, or derivative cells such as epithelial cell line engineered to express, or to have reduced expression of, specific transporter proteins, such as MDR1, rodent mdr1 a or b, BCRP, MRP2, or OATPB 1, among others. In a preferred aspect, the derivative cells are polarized epithelial cells genetically engineered to have a reduced level of BCRP expression. In a more preferred aspect, the derivative cells are Caco-2 cells expressing a form of RNA that reduces the expression of BCRP. In one preferred aspect, the derivative cells are polarized epithelial cells genetically engineered to have a reduced level of PGP expression. In a more preferred aspect, the derivative cells are Caco-2 cells expressing a form of RNA that reduces the expression of PGP. In another preferred aspect, the derivative cells are polarized epithelial cells genetically engineered to have a reduced level of MRP2 expression. In a more preferred aspect, the derivative cells are Caco-2 cells expressing a form of RNA that reduces the expression of MRP2. In another preferred aspect, the derivative cells are polarized epithelial cells genetically engineered to express MDR1. In a more preferred aspect, the derivative cells are MDCK cells expressing MDR1. As will now be apparent to those of skill in the art, any number of cell lines that are found to persist using the methods described herein will also lend themselves to being received in a manner analogous to the methods described herein, such embodiments are considered to be within the scope of the described methods.

Described herein is a kit for transporting a cell culture grown on a tissue culture plate having a permeable tissue culture plate insert therein, said permeable tissue culture plate insert providing the tissue culture plate with an apical chamber and a basolateral chamber, wherein cells are deposited on the permeable tissue culture insert, wherein the apical chamber of the tissue culture plate is essentially free of tissue culture medium and the basolateral chamber of the tissue culture plate contains a solidifiable form of tissue culture medium. Also described are methods for using the kit to transport a cell culture on a tissue culture plate having a permeable tissue culture plate insert therein, said permeable tissue culture plate insert providing the tissue culture plate with an apical chamber and a basolateral chamber, wherein cells are deposited on the permeable tissue culture insert, wherein the apical chamber of the tissue culture plate is essentially free of tissue culture medium and the basolateral chamber of the tissue culture plate contains a solidifiable form of tissue culture medium. The kits described herein can also include instructions for how to transport, handle, or feed the cell cultures of the kits. In addition, the described kits may have tissue culture medium for use with the cell cultures of the kits.

### EXAMPLES

### Cell Lines

Four polarized cell lines were studied for their ability to withstand being shipped in solidified medium: Madin-Darby canine kidney (MDCK) cells, MDR-MDCK cells, Caco-2 cells, and C2BBe1 cells. MDCK cells (ATCC accession number CCL-34) are kidney cells from a normal adult female cocker spaniel. These cells form polarized monolayers connected by tight junctions. MDCK cells are a well-known model system used to study the biology of polarized epithelial cell monolayers.

The MDR-MDCK cell line was obtained from the NIH. The MDR-MDCK cells are MDCK cells transfected with the human MDR1 gene. Pastan, et al., Proc. Natl. Acad. Sci. U.S.A. June 1988;85(12):4486-90. These polarized cells overexpress human P-glycoprotein (PGP) almost exclusively on the apical plasma membrane and are useful in identifying and characterizing PGP substrates. Pastan, et al.

The Caco-2 cell lines (ATCC Accession Number HTB-37™) is tumor cell line derived from a human colon tissue sample of a patient with colorectal adenocarcinoma. This cell line is widely used in studies relating to epithelial biology due to its ability to form polarized cell monolayers and accept foreign DNA by transfection.

The C2BBe1 cell line (ATCC Accession Number CRL-2102) was derived from the Caco-2 cell line in 1988 by limiting dilution. The clone was selected on the basis of morphological homogeneity and exclusive apical villin localization. C2BBe1 cells form a polarized monolayer with an apical brush border (BB) morphologically comparable to that of the human colon. Isolated BB contain the microvillar proteins villin, fimbrin, sucrase-isomaltase, BB myosin-1, and the terminal web proteins fodrin and myosin II. The cells express substantial levels of BB mysosin I similar to that of the human enterocyte. Although clonal, and far more homogenous than the parental Caco-2 cell line with respect to BB expression, these cells are still heterogeneous for microvillar length, microvillar aggregation, and levels of expression of certain BB proteins.

### Cell Culture Conditions

All cell lines were seeded on Transwell® tissue culture plate inserts (Coming, Inc., Corning, N.Y.) and allowed to form monolayers prior to shipping experiments. Transwell® inserts containing monolayers of cells were prepared as follows: each insert of a 12-well Transwell® insert was pretreated with rat tail collagen to promote cell attachment. Then, 1.5 mL of cell culture media (90% Dulbecco's Modified Eagle Medium supplemented with 10% fetal bovine serum) was added to the bottom wells of a 12 well Transwell® insert. The cells were detached from stock T-150 tissue culture flasks by trypsinization, and resuspended in cell culture media. Clumps of cells were broken up by repeated pipetting to generate a uniform suspension of cells. The number of cells in suspension was counted using a hemocytometer. Supplemental cell culture medium was added to the cell suspension to bring the cell count to approximately 136,000 cells per mL. Approximately 68,000 cells, were added to each apical chamber of the 12 well Transwell® insert. The cells were allowed to attach overnight and fed with fresh cell culture medium the following day by adding 0.5 mL of medium to the apical chamber and 1.5 mL of medium to the basolateral chamber of each well. Medium was changed every other day for at least 15 days for the Caco-2 and C2BBe1 cell lines or 6 days for the MDR-MDCK and MDCK cell lines prior to selecting monolayers for shipping. Up to one or two dozen 12-well Transwell® inserts were prepared at one time from the same parent stock flask. Any cells not used for seeding Transwell® inserts were recultured in T-150 stock tissue culture flasks.

### Cell Transport

All cell lines were prepared for shipping as follows: first, a 4% agarose solution was prepared using a microwave oven to melt 0.4 g of agarose (L.M.P. Ultrapure Agarose, Invitrogen catalog number 15517, lot number 1065345) into 10 mL of pre-warmed 37° C. 1× Dulbecco's Phosphate Buffered Saline (Invitrogen catalog number 14190). While in liquid form, the 4% agarose solution was diluted in tissue culture medium to yield a 0.75% agarose medium solution (for example 3 mL of 4% agarose was added to 13 ml of media). Standard Dulbecco's Modified Eagle's Medium (DMEM) containing 10% fetal bovine serum was used. This 0.75% agarose-medium was used directly, or could be kept liquid in a 37° C. water bath. One milliliter of 0.75% agarose-medium was added to the basolateral chamber of each monolayer after media in the apical and basolateral chambers of each Transwell® device was removed by aspiration. The plates were then placed at 4° C. until the agarose medium solidified (approximately thirty minutes). The plates were then wrapped in Parafilm® and packaged so that they would not move during shipping. A temperature recorder was also included in the shipping container to record temperature during shipping. Cells were then driven to FedEx Kinko's® 4120 Concord Pike, Wilmington, Del. 19803, where they were dropped off, and FedEx® returned the cells the following morning to Absorption Systems LP, Oakland's Corporate Center, 436 Creamery Way, Suite 600, Exton, Pa. 19341-2556.

After receiving the cells, the plates were unwrapped and fed on both the apical and basolateral sides with pre-warmed complete DMEM, the agarose medium still remained in the basolateral chambers. The plates were then placed in a cell culture incubator at 37° C. to soften the agarose medium. After at least two hours, the Transwells® inserts were moved from the plates in which they were shipped to new tissue culture plates, were fed again, and allowed to recover. MDR-MDCK and MDCK cells required only overnight to recover. C2BBe1 cells required at least two days of recovery before a quality control assay was preformed.

Quality control testing of a batch of Transwell® inserts was carried out as follows: The monolayers shipped in agarose and at least six monolayers that were not shipped, but rather were maintained under normal growth conditions in an incubator, were selected for quality control assessment. The cells were placed in a blank Transwell® tissue culture plate containing Hank's Balanced Salt Solution (HBSS) pH 7.4 containing 10 mM HEPES and 15 mM glucose (HBSSg) pre-warmed to 37° C. The medium was aspirated from the wells and HBSSg was used to rinse the cell monolayers on the inserts. Fresh HBSSg was added after the monolayers were washed, the inserts were removed from the assay plate, and the transepithelial electrical resistance (TEER) value of the monolayers was determined using an ENDOHM transepithelial electrical resistance measurement apparatus (World Precision Instruments).

C2BBe1cell monolayers having a TEER value above 450 Ω·cm² and MDCK cell monolayers having a TEER value above 1200 Ω·cm² are considered acceptable for use in permeability studies. Studies were performed if cells were at, or only slightly below, acceptable TEER values. Cells from each batch were tested for permeability of reference compounds as follows: The following pre-warmed quality control solution was added to the apical chamber of Transwell® inserts: 0.5 mM lucifer yellow, 10 μM atenolol, 10 μM propranolol, 5 μM estrone-3-sulfate, and 10 μM digoxin in Hanks Balanced Salt Solution (HBSS) containing 10 mM HEPES and 15 mM glucose (HBSSg), at pH 7.4±0.2. The basolateral chambers contained HBSSg pre-warmed to 37° C. Transwell® inserts were placed in a humidified incubator and incubated for 2 hours at 37° C. in an atmosphere containing 5% CO₂. Following incubation, samples were taken from the basolateral chamber for analysis of lucifer yellow content by fluorescence detection, and the other compounds by LC/MS/MS.

Permeability values were calculated from the donor (apical chamber) concentrations and net increases in receiver (basolateral chamber) concentrations at the 2 hour sampling interval. The permeability for the time interval, Papp (t1−t2), was calculated according to the following formula: Papp (t1−t2)=((Ct2−Ct1)/(t2−t1))×Vr/(A×Cd). Ct2−Ct1 is the cumulative concentration difference in the receiver (basolateral) compartment at each time interval in μM, (in this example Ct1 is assumed to be “0” because the entire time interval (120 min) is used in the calculation); Vr is the volume of the receiver compartment (in cm³); A is the area of the cell monolayer (1.13 cm² for 12-well Transwell®), and Cd is concentration in the donor sample compartment (apical chamber) in μM, which is equal to the concentration in the donor solution described above. The apparent permeability for each compound used for quality control purposes was the average of all Papp values calculated for all replicates tested, typically 3 replicate inserts per assay condition.

This assay was repeated by adding the dosing solution with the quality control compounds to the basolateral chamber of the Transwell® and measuring the digoxin and estrone-3-sulfate (E3S) concentrations in the apical well after 2 hours. A calculated Papp for digoxin in this assay direction should be at least 3 times higher than that calculated for the apical to basolateral direction is an indication of functional expression of PGP in the cell monolayers. MDCK monolayers should have an apical concentration of digoxin less then 3 times higher than that calculated value for the basolateral chamber as these cells lack high levels of functional PGP. A calculated Papp for E3S in the apical chamber should be 4 times higher than that calculated for the basolateral chamber to indicate functional expression of BCRP in C2BBe1 cell monolayers. Both the MDCK and MDR-MDCK cell lines should have an apical concentration of E3S 4 times higher than that of the basolateral chamber due to lack of high levels of functional BCRP.

The permeability of lucifer yellow, propranolol, and atenolol, cell monolayer integrity marker compounds, were also measured for each monolayer to determine whether monolayer integrity was impaired during the permeation study. The transport assay buffer was HBSSg at pH 7.4±0.1. Compound dosing solutions were prepared in HBSSg from DMSO stock with 1% DMSO final concentration. Cell monolayers used for the studies were washed twice with HBSSg, and the transepithelial electrical resistance (TEER) was measured for each membrane. For apical to basolateral transport, 0.5 mL dosing solution was added to the apical chamber, and 1.5 mL of HBSSg was added to the basolateral chamber. For basolateral to apical transport, 1.5 mL dosing solution was added to the basolateral chamber, and 0.5 mL of HBSSg was added to the apical chamber. Then the cell monolayers were incubated at 37° C. (5% CO₂) in a humidified incubator for 120 minutes. Each determination was performed in triplicate. Samples were taken from the receiver compartment at 120 minutes. The acceptable membrane integrity and efflux transporter expression criteria were: propranolol permeability 15-25×10⁻⁶ cm/s, lucifer yellow permeability≦0.4×10⁻⁶ cm/s, atenolol permeability≦0.5×10⁻⁶ cm/s. Lucifer yellow was measured by florescence assay ad all other compounds were assayed by LC/MS using electrospray ionization as summarized below.

### Summary of LC/MS Analytical Methods

A liquid chromatography instrument capable of generating a gradient of eluting buffer (mobile) phase was used. A chromatography column (Keystone Hypersil BDS C18 30×2.0 mm i.d., 3 μm, with guard column) connected to the liquid chromatography instrument was used to analyze a 10 μL sample of buffer from the transport assay. Two mobile phases were continuously mixed in various proportions to establish a compositional gradient. Typical mobile phases used for this assay were an aqueous buffer, such as 25 mM ammonium formate buffer, pH 3.5, and an organic solvent, such as acetonitrile. The elution gradient was formed by mixing appropriate proportions of mobile phases from two mobile phase reservoirs. In the example listed below, one reservoir contained the aqueous buffer and the second reservoir contained a mixture of acetonitrile and aqueous buffer in the proportion of 9:1 (volume:volume). The gradient program in the liquid chromatography instrument can be set to form a variety of gradients from linear, in which the composition changes from buffer to acetonitrile plus buffer at a fixed rate, to ballistic, in which the composition changes suddenly from buffer to acetonitrile plus buffer at a specific time in the analysis. Gradient program conditions for the analysis used herein are listed in Table 1 below, in which %A refers to the fraction of aqueous buffer in the gradient and % B refers to the fraction of acetonitrile buffer mixture in the gradient. The time column refers to the time after the sample was injected with 0.0 minutes being the sample injection point. In this example the gradient was of the ballistic type, suddenly changing composition at 1.5 minutes after sample injection.

TABLE 1
Liquid Chromatography Instrument Gradient Program Conditions

| Time (Min) | % A | % B |
|---|---|---|
| 0.0 | 100 | 0 |
| 0.5 | 100 | 0 |
| 1.5 | 0 | 100 |
| 2.0 | 0 | 100 |
| 2.1 | 100 | 0 |
| 3.5 | 100 | 0 |

The liquid chromatography instrument autosampler syringe was rinsed with 0.2% formic acid in water/acetonitrile/2-propanol: 1/1/1 (v/v/v) between injections. The eluant from the chromatographic column was directed to the electrospray interface of a triple quadropole mass spectrometer (MS/MS), where the solvent and buffer were evaporated, and compounds eluted from the chromatographic column were ionized to form positive or negative ions.

In the examples below an instrument, typically a PE SCIEX API 2000, 3000 or in some cases a 4000 model was used to separate and detect the ions. Triple quadropole instruments, such as these, can separate parent ions using the first quadropole magnetic, fragment them in the second quadropole chamber and detect specific fragments of the parent ion using the third quadropole to focus ions of a pre-specified mass onto the instrument's detector. This mode of detection is frequently referred to as Multiple Reaction Monitoring (MRM). MRM permits very specific and sensitive detection of compounds of interest with mass resolutions of at least ±1 atomic mass units and limits of detection in the nanogram per milliliter range. Typical parent and fragment ions used for detection of the compounds mentioned in the examples are presented in Table 2.

TABLE 2
Parent and fragment ions used for detection

| Compound | Q1/Q3 |
|---|---|
| Atenolol | +267.4/145.2 |
| Propanolol | +260.4/116.2 |
| Estrone-3-Sulfate | −349.2/269.1 |
| Digoxin | +798.6/651.5 |

Where Q1 refers to the mass selection setting of the first quadropole magnet and Q3 refers to the mass selection setting of the second quadropole magnet. The “+” or “−” sign refers to the sign of the charge on the ions being monitored.

Another parameter that can be adjusted on these mass spectrometers is the dwell time, which refers to the time period in which the two quadropoles are set to select and detect a particular combination of parent and fragment (daughter) ions. Multiple compounds can be detected in the same chromatographic analysis by appropriate adjustment of the chromatographic conditions and the mass spectrometer dwell times. Typical dwell times range from about 10 to about 100 milliseconds per ion pair combination. Skilled analysts can usually determine a combination of chromatographic conditions and dwell times that will allow detection and quantification of up to about 6 compounds in the same sample, provided that their ion masses differ by at least 5 atomic mass units.

Analytical standards with concentrations ranging from about 1 ng/mL up to about 1,000 ng/mL were prepared in the same matrix as used for transport assay samples. A standard curve was prepared by plotting the MS/MS detector response versus the standard concentration. The standard curve was fitted to a linear or polynomial response curve using software provided by the instrument manufacturer. The concentration of compound in the unknowns was determined by back calculating from the detector response. Alternatively, the ratio of detector responses between the compounds of interest and a reference standard compound added to the standards and samples at a fixed concentration is used to construct the standard curve and quantify unknowns. This is known as the internal standard method of sample quantification.

The following examples are provided to enhance the understanding of the subject matter disclosed herein. They are intended to provide exemplary illustrations, not to limit, the disclosed subject matter.

Applicants specifically incorporate the entire contents of all cited references in this disclosure. Further, when an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### Example 1

### Shipment of Polarized Cell Monolayers Having Agarose-Supplemented Medium in the Apical Chamber of Transwell Inserts

Initially, MDR-MDCK cells were shipped with 0.25% agarose medium in the apical chamber only or in both the apical and basolateral chambers of Transwell® tissue culture plates. Following shipment, the agarose medium was removed and the integrity of cell monolayers was assessed by measuring TEER and passive diffusion rates. Cell monolayers were considered intact if the TEER value was greater than 1200 Ω·cm² and lucifer yellow values were less than 0.8.×10⁻⁶ cm/s. The shipping procedure did not appear to disrupt cell monolayer integrity, whether agarose medium was added to the apical and basolateral chambers of a Transwell® or only to the apical chamber, as there were almost no TEER failures and few lucifer yellow failures (Table 3). In light of these results, it was decided that agarose should be added to the apical chamber only, as it is easier to handle the cell monolayers in this state rather than with agarose medium in both the apical and basolateral chambers of Transwell® inserts. Although the cells were viable after shipping, 0.25% agarose did not withstand shipping intact; therefore, the optimal shipping conditions still needed to be determined.

TABLE 3
Preliminary conditions for agarose shipping with MDR-MDCK Cells.
Summary

| | Shipped | Control |
|---|---|---|
| cell type | MDR-MDCK | MDR-MDCK |
| Seed date | Oct. 18, 2007 | Oct. 18, 2007 |
| passage # | 16 | 16 |
| Days in culture when shipped | 6 D | N/A |
| Days in culture when assayed | 8 D | 8 D |

| | TEER Data | | L.Y. Papp Data | |
|---|---|---|---|---|
| | TEER | | L.Y. Papp | |
| Method | (Ω · cm²) | Average | (cm/s) 10⁻⁶ | Average |
| Control (cells maintained in | 1259.95 | 1073.9 | 0.09 | 0.12 |
| normal medium and | 1022.65 | | 0.05 | |
| unshipped) | 219.22 | | 0.13 | |
| | 1632.85 | | 0.13 | |
| | 1426.06 | | 0.17 | |
| | 882.53 | | 0.15 | |
| Agarose medium in apical | 2513.12 | 2497.3 | 0.19 | 0.40 |
| chamber only | 2275.82 | | 0.16 | |
| | 2101.80 | | 0.31 | |
| | 2660.02 | | 0.31 | |
| | 2490.52 | | 0.49 | |
| | 2942.52 | | 0.92 | |
| Agarose medium in apical | 2321.02 | 2173.2 | 0.09 | 0.12 |
| and basolateral chambers | 2004.62 | | 0.04 | |
| | 1222.66 | | 0.08 | |
| | 2343.62 | | 0.15 | |
| | 2170.73 | | 0.18 | |
| | 2976.42 | | 0.18 | |

### Example 2

### Determination of the Optimal Percentage of Agarose-Supplemented Medium to Use for Shipping Polarized Cell Monolayers

Polarized MDR-MDCK cells were prepared by applying a range of agarose-media formulations to the apical surface of cells before being shipped, as described previously, to determine the percentage of agarose needed to produce media optimal for use in shipping cells on Transwell® inserts. Following shipment, the agarose medium was removed and the integrity of cell monolayers was assessed by measuring TEER and passive diffusion rates. TEER and post-shipment lucifer yellow values were used to evaluate the integrity of cell monolayers. Cell monolayers were considered intact if the TEER value was greater than 1200 Ω·cm² and lucifer yellow values were less than 0.8.×10⁻⁶ cm/s. Agarose percentages less then 0.5% were found to not withstand shipping conditions intact, consistent with prior findings. Cells covered with media supplemented with greater than 0.5% agarose shipped well; however, the agarose-supplemented media was difficult to remove from the apical chamber of Transwell® inserts, which caused TEER and lucifer yellow failures due to damaged cell monolayers. Media supplemented with 0.5% agarose was selected as the optimal shipping medium, as it could be removed from Transwell® inserts without significantly damaging cell monolayers while also providing durability during shipment (Table 4).

TABLE 4
Ascertaining the correct percentage of agarose to use for shipping.
Summary

| | Shipped | Unshipped |
|---|---|---|
| cell type | MDR-MDCK | MDR-MDCK |
| Seed date | Nov. 5, 2007 | Nov. 5, 2007 |
| passage # | 21 | 21 |
| Days in culture when shipped | 7 | N/A |
| Days in culture when assayed | 9 | 9 |

| | TEER Data | | L.Y. Papp Data | |
|---|---|---|---|---|
| | TEER | | L.Y. Papp | |
| Method | (Ω · cm²) | Average | (cm/s) 10⁻⁶ | Average |
| Control | 1415.89 | 1427.6 | 0.19 | 0.10 |
| | 1839.64 | | 0.06 | |
| | 1930.04 | | 0.11 | |
| | −11.10 | | 0.07 | |
| | 1884.84 | | 0.06 | |
| | 1506.29 | | 0.10 | |
| Agarose 0.25% | 2093.89 | 2435.3 | 0.07 | 0.07 |
| | 2382.04 | | 0.07 | |
| | 2596.74 | | 0.06 | |
| | 2074.68 | | 0.09 | |
| | 2743.64 | | 0.05 | |
| | 2721.04 | | 0.08 | |
| Agarose 0.5% | 2528.94 | 2048.7 | 0.14 | 0.58 |
| | 1633.98 | | 0.23 | |
| | 369.51 | | 2.37 | |
| | 2834.04 | | 0.16 | |
| | 3093.94 | | 0.11 | |
| | 1831.73 | | 0.48 | |
| Agarose 0.75% | 1997.84 | 2096.5 | 2.37 | 0.56 |
| | 1430.58 | | 0.19 | |
| | 3263.44 | | 0.13 | |
| | 2980.94 | | 0.04 | |
| | 1954.90 | | 0.13 | |
| | 951.46 | | 0.49 | |
| Agarose 1% | 2980.94 | 2534.8 | 0.11 | 0.46 |
| | 729.98 | | 0.59 | |
| | 3545.94 | | 0.13 | |
| | 600.03 | | 1.51 | |
| | 3466.84 | | 0.11 | |
| | 3884.94 | | 0.33 | |
| Agarose 2% | 689.30 | 446.5 | 0.94 | 1.79 |
| | 120.91 | | 2.37 | |
| | 212.44 | | 2.37 | |
| | 1317.58 | | 0.40 | |
| | 352.56 | | 2.26 | |
| | −13.56 | | 2.37 | |

### Example 3

### Cell Monolayer Integrity is not Disrupted by Transport with Agarose-Medium in Only the Apical or Only the Basolateral Chamber of a Tissue Culture Plate

MDR-MDCK cells were seeded onto Transwell® inserts and cultured as described previously. Prior to shipping, media was removed from the apical and basolateral chambers of the Transwell® insert and the apical medium was replaced with 0.5% agarose medium. Following shipping, four different people aspirated the apical shipping medium and fed the cells to demonstrate the post-shipment integrity of the cell monolayers was not dependant on a particular tissue culture handling technique. Monolayer integrity was assessed using TEER and lucifer yellow assays as described previously (Table 5). The numbers above the cell line indicate the person who aspirated and fed the cells. While the cells fed by persons two and four did not reflect acceptable average TEER values they passed all other quality control criteria. The data suggests the majority of cell monolayers shipped with agarose-medium in only the apical chamber remained intact regardless of the person handling the cells following shipment.

Cells were also shipped with 0.5% agarose medium in only the basolateral chamber of the tissue culture well, to assess the ability of MDR-MDCK monolayers to withstand shipping in the absence of apical medium. Following removal of growth medium, one milliliter of 0.5% agarose medium was placed in the basolateral chamber of the tissue culture well and no medium was added to the apical chamber. The cells were shipped along with the MDR-MDCK cells described above. Following shipping, the cells were fed and after two hours moved to a new tissue culture plate containing growth medium in the basolateral chamber. Monolayer integrity was assessed using TEER and lucifer yellow assays as described previously (Table 5). The cells with the agarose on the basolateral side survived shipping as well as, if not better than, cells with agarose on the apical side.

TABLE 5
Cell monolayer integrity is not disrupted by transport with agarose-medium in
only the apical or only the basolateral chamber of a Transwell ® tissue culture plate.
Summary

| | Apical | Apical | Apical | Apical |
|---|---|---|---|---|
| cell type | MDR-MDCK | MDR-MDCK | MDR-MDCK | MDR-MDCK |
| Seed date | Feb. 13, 2008 | Feb. 13, 2008 | Feb. 13, 2008 | Feb. 13, 2008 |
| passage # | 11 | 11 | 11 | 11 |
| Days in culture when shipped | 5 D | 5 D | 5 D | 5 D |
| Days in culture when assayed | 7 D | 7 D | 7 D | 7 D |
| Average TEER (Ω · cm²), n = 6 | 1508 | 1507.80 | 1191.96 | 1191.96 |
| Average LY Papp (cm/s) | 0.20 | 0.20 | 0.19 | 0.19 |
| Passive diffusion, Papp (cm/s, 10⁻⁶, n = 3) | | | | |
| Atenolol A->B | 0.20 | 0.11 | 0.26 | 0.14 |
| Propranolol A->B | 20.49 | 16.40 | 24.21 | 17.60 |
| Pgp transport, Papp (cm/s, 10⁻⁶, n = 3) | | | | |
| Digoxin A->B | 0.11 | 0.06 | 0.17 | 0.07 |
| Digoxin B->A | 5.24 | 6.19 | 4.65 | 6.39 |
| Efflux ratio | 47.86 | 104.53 | 26.59 | 90.48 |
| | Apical | Apical | Basolateral | Control |
| cell type | MDR-MDCK | MDR-MDCK | MDR-MDCK | MDR-MDCK |
| Seed date | Feb. 13, 2008 | Feb. 13, 2008 | Feb. 13, 2008 | Feb. 13, 2008 |
| passage # | 11 | 11 | 11 | 11 |
| Days in culture when shipped | 5 D | 5 D | 5 D | — |
| Days in culture when assayed | 7 D | 7 D | 7 D | 7 D |
| Average TEER (Ω · cm²), n = 6 | 1405 | 1199.12 | 1391.22 | 1368.05 |
| Average LY Papp (cm/s) | 0.89 | 0.32 | 0.07 | 0.26 |
| Passive diffusion, Papp (cm/s, 10⁻⁶, n = 3) | | | | |
| Atenolol A->B | 0.31 | 0.13 | 0.10 | 0.31 |
| Propranolol A->B | 25.81 | 26.36 | 18.63 | 26.61 |
| Pgp transport, Papp (cm/s, 10⁻⁶, n = 3) | | | | |
| Digoxin A->B | 0.18 | 0.09 | 0.06 | 0.10 |
| Digoxin B->A | 7.05 | 8.27 | 6.09 | 6.70 |
| Efflux ratio | 38.48 | 92.30 | 95.22 | 69.16 |

An additional experiment was performed to confirm the results of shipping DMR-MDCK cells with medium only in the basolateral chamber of the Transwell® plate. The experimental procedure was exactly the same as the first time the cells were shipped with medium only in the basolateral chamber of the Transwell® plate. Again, the cell monolayers retained their integrity following shipment (Table 6). Based on these results and the increased ease of handling the cells, agarose was used exclusively on the basolateral chamber for future shipping experiments.

TABLE 6
MDR-MDCK cell monolayer integrity is not disrupted by transport
with agarose-medium in only the basolateral chamber of
a Transwell ® tissue culture plate.
Summary

| | Shipped | Control |
|---|---|---|
| Cell type | MDR-MDCK | MDR-MDCK |
| Seed date | Mar. 5, 2008 | Mar. 5, 2008 |
| passage # | 16 | 16 |
| Days in culture when shipped | 5 D | — |
| Days in culture when assayed | 10 D | 10 D |
| Average TEER (Ω · cm²), n = 6 | 1432.18 | 1335 |
| Average LY Papp (cm/s) | 0.10 | 0.04 |
| Passive diffusion, Papp | | |
| (cm/s, 10⁻⁶, n = 3) | | |
| Atenolol A->B | 0.33 | 0.18 |
| Propranolol A->B | 17.80 | 17.28 |
| Pgp transport, Papp (cm/s, 10⁻⁶, n = 3) | | |
| Digoxin A->B | 0.21 | 0.15 |
| Digoxin B->A | 9.14 | 10.84 |
| Efflux ratio | 42.99 | 70.82 |

### Example 4

### C2BBe1 Cell Monolayer Integrity is Not Disrupted by Transport with Agarose-Medium in Only Basolateral Chamber of a Tissue Culture Plate

C2BBe1 were shipped with 0.5% agarose medium in only the basolateral chamber of the Transwell® plate to determine if this method of shipping would allow C2BBe1 cell monolayers to remain intact following shipment. The agarose and shipping conditions were the same for the C2BBe1 cells as described for the MDR-MDCK cells. Two different seed dates of the C2BBe1 cell line were shipped but both were assayed on the same day. Monolayer integrity was assessed using TEER and lucifer yellow assays as described previously. Cell monolayers seeded on Transwells® 21 days prior to shipping did not seem to perform as well as the younger cells, seeded onto Transwells® only 15 days before shipping, in terms of cell monolayer integrity. The results indicate that C2BBe1 cell monolayers remained intact and that younger cell seemed to tolerate shipping better than older cells (Table 7).

In addition, MDR-MDCK were also shipped having 0.5% agarose medium in only the basolateral chamber. This experiment provides a third demonstration of MDR-MDCK cell monolayers being shipped with agarose on the basolateral side only. All agarose and shipping conditions remained the same. Monolayer integrity was assessed using TEER and lucifer yellow assays as described previously. As before, MDR-MDCK cell monolayer integrity was not compromised by the shipping process (Table 7).

TABLE 7
C2BBe1 and MDR-MDCK cell monolayer integrity is intact following
shipping with agarose-media in only the basolateral well
of a Transwell® tissue culture plate.
Summary

| | Shipped | Shipped | Control |
|---|---|---|---|
| cell type | C2BBe1 | C2BBe1 | C2BBe1 |
| Seed date | Mar. 5, | Mar. 11, | Mar. 5, |
| | 2008 | 2008 | 2008 |
| passage # | 66 | 65 | 66 |
| Days in culture when shipped | 21 D | 15 D | — |
| Days in culture when assayed | 27 D | 21 D | 27 D |
| Average TEER (Ω · cm²), n = 6 | 370 | 605.49 | 682.52 |
| Average LY Papp (cm/s) | 0.16 | 0.32 | 0.24 |
| Passive diffusion, Papp | | | |
| (cm/s, 10⁻⁶, n = 3) | | | |
| Atenolol A->B | 0.36 | 0.24 | 0.31 |
| Propranolol A->B | 16.32 | 21.06 | 23.39 |
| Pgp transport, Papp | | | |
| (cm/s, 10⁻⁶, n = 3) | | | |
| Digoxin A->B | 0.32 | 0.58 | 0.23 |
| Digoxin B->A | 6.86 | 10.26 | 5.67 |
| Efflux ratio | 21.68 | 17.61 | 25.00 |
| BCRP transport, Papp | | | |
| (cm/s, 10⁻⁶, n = 3) | | | |
| E3S A->B | 0.40 | 0.27 | 0.48 |
| E3S B->A | 6.27 | 8.95 | 5.45 |
| Efflux ratio | 15.67 | 33.07 | 11.38 |

| | Shipped | Control |
|---|---|---|
| cell type | MDR-MDCK | MDR-MDCK |
| Seed date | Mar. 20, 2008 | Mar. 20, 2008 |
| passage # | 17 | 17 |
| Days in culture when shipped | 6 | — |
| Days in culture when assayed | 12.00 | 12.00 |
| Average TEER (Ω · cm²), n = 6 | 1683 | 1368.52 |
| Average LY Papp (cm/s) | 0.10 | 0.12 |
| Passive diffusion, Papp | | |
| (cm/s, 10⁻⁶, n = 3) | | |
| Atenolol A->B | 0.08 | 0.12 |
| Propranolol A->B | 14.04 | 17.69 |
| Pgp transport, Papp (cm/s, 10⁻⁶, n = 3) | | |
| Digoxin A->B | 0.09 | 0.15 |
| Digoxin B->A | 6.40 | 6.37 |
| Efflux ratio | 69.61 | 42.33 |
| BCRP transport, Papp | | |
| (cm/s, 10⁻⁶, n = 3) | | |
| E3S A->B | 0.10 | 0.17 |
| E3S B->A | 0.10 | 0.13 |
| Efflux ratio | 0.99 | 0.75 |

### Example 5

### Determination of the Optimal Percentage of Agarose-Supplemented Medium to Use for Shipping Polarized Cell Monolayers Having Medium in Only the Basolateral Chamber of a Transwell® Tissue Culture Plate

After repeating shipping experiments multiple times with agarose medium only on the basolateral side successfully, it was found that the 0.5% agarose medium did not always remain adherent to the well surface. Since the agarose medium did not need to be aspirated from the basolateral chamber of the Transwell® plate after shipping, unlike when it was located in the apical chamber, medium containing higher concentrations of agarose could be assessed. Shipping experiments were conducted using 0.5, 0.75, and 1% agarose medium in the basolateral chamber of the tissue culture plates (Table 8). The results suggest that none of these concentrations of agarose compromised cell monolayer integrity. Medium having 0.75 or 1% agarose medium both worked well for shipping cells. Future experiments were performed having 0.75% agarose medium in the basolateral chamber, as this concentration of agarose provided a more economically sound option than 1% agarose.

TABLE 8
Ascertaining the correct percentage of agarose-medium to use for shipping.
Summary

| | 1% agarose | 0.75% agarose | 0.5% agarose | 0% agarose | Control |
|---|---|---|---|---|---|
| cell type | C2BBe1 | C2BBe1 | C2BBe1 | C2BBe1 | C2BBe1 |
| Seed date | Apr. 1, 2008 | Apr. 1, 2008 | Apr. 1, 2008 | Apr. 1, 2008 | Mar. 27, 2008 |
| passage # | 68 | 68 | 68 | 68 | 66 |
| Days in culture when shipped | 16 D | 16 D | 16 D | 16 D | — |
| Days in culture when assayed | 21 D | 21 D | 21 D | 21 D | 26 D |
| Average TEER (Ω · cm²), n = 6 | 706 | 686 | 613 | 394 | 889 |
| Average LY Papp (cm/s) | 0.21 | 0.17 | 0.14 | 0.88 | 0.62 |
| Passive diffusion, Papp (cm/s, 10⁻⁶, n = 3) | | | | | |
| Atenolol A->B | 0.30 | 0.18 | 0.16 | 1.39 | 0.36 |
| Propranolol A->B | 21.77 | 21.61 | 27.35 | 27.27 | 19.24 |
| Pgp transport, Papp (cm/s, 10⁻⁶, n = 3) | | | | | |
| Digoxin A->B | 0.50 | 0.32 | 0.41 | 0.94 | 0.44 |
| Digoxin B->A | 6.92 | 7.97 | 7.61 | 6.96 | 7.30 |
| Efflux ratio | 13.81 | 25.19 | 18.59 | 7.43 | 16.61 |

### Example 6

### MDCK Cell Monolayer Integrity is not Disrupted by Transport with Agarose-Medium in Only the Basolateral Chamber of a Transwell® Tissue Culture Plate

A shipping experiment was performed using MDCK cells to determine whether these cells could be shipped with agarose medium only in the basolateral chamber of a Transwell® plate. The experimental method was exactly the same as described for the other two cell lines. Following shipment cell monolayer integrity was assessed using TEER and lucifer yellow assays. The cell monolayers for the shipped and control MDCK cells remained intact (Table 9). Therefore, these data indicate that multiple adherent cell lines can be shipped with agarose medium located only on the basolateral side of the Transwell®.

TABLE 9
MDCK cells shipped with 0.75% agarose media only in the basolateral
chamber of the Transwell® tissue culture plate.
Summary

| | Shipped | control |
|---|---|---|
| cell type | MDCK | MDCK |
| Seed date | May 26, 2008 | May 26, 2008 |
| passage # | 12 | 12 |
| Days in culture when shipped | 7.00 | 7.00 |
| Days in culture when assayed | 10.00 | 10.00 |
| Average TEER (Ω · cm²), n = 6 | 4819.83 | 8712.68 |
| Average LY Papp (cm/s) | 0.13 | 0.27 |
| Passive diffusion, Papp | | |
| (cm/s, 10⁻⁶, n = 3) | | |
| Atenolol A->B | 0.10 | 0.31 |
| Propranolol A->B | 14.49 | 14.05 |
| Pgp transport, Papp (cm/s, 10⁻⁶, n = 3) | | |
| Digoxin A->B | 0.04 | 0.20 |
| Digoxin B->A | 3.83 | 0.94 |
| Efflux ratio | 102 | 4.69 |
| BCRP transport, Papp | | |
| (cm/s, 10⁻⁶, n = 3) | | |
| E3S A->B | 0.13 | 0.26 |
| E3S B->A | 0.16 | 0.06 |
| Efflux ratio | 1.18 | 0.22 |

### Example 7

### MDR-MDCK Cell Monolayer Integrity is not Disrupted by Air Transport with Agarose-Medium in Only Basolateral Chamber of a Transwell® Tissue Culture Plate

MDR-MDCK cells were used to determine whether cells having 0.75% agarose media in only the basolateral chamber of a Transwell® plate could be shipped by air transit. MDR-MDCK cells were seeded onto Transwell® inserts, as described previously. Prior to shipping the cells, growth media was removed from the apical and basolateral chambers of the Transwell® plate and 1.5 mL of 0.75% agarose medium was placed in the basolateral chamber. The medium was allowed to solidify and the cells were packaged for shipping, as described previously. The packaged cells were shipped from Exton, Pa. to Folsom, Calif. and then returned to Exton, Pa. In all the cells spent three days in transit, making two cross-country fights, without being fed or otherwise maintained. Upon receipt, the integrity of the cell monolayers was assessed relative to control MDR-MDCK cells that were maintained under normal growth conditions (Table 10). These results indicate that neither air transit nor extended shipping times disrupt cell monolayer integrity when cells are shipped with agarose medium located only on the basolateral side of the Transwell®. Furthermore, there results indicate that the cell monolayers remain intact following approximately three days outside of a tissue culture incubator without apical tissue culture medium.

TABLE 10
Air cargo shipment of MDR-MDCK cells with 0.75% agarose media
only in the basolateral chamber of Transwell® tissue culture inserts.
Summary

| | Shipped | Control |
|---|---|---|
| cell type | MDR-MDCK | MDR-MDCK |
| Seed date | Jul. 2, 2008 | Jul. 2, 2008 |
| passage # | 34 | 34 |
| Days in culture when shipped | 6 | N/A |
| Days in culture when assayed | 12 | 12 |
| Average TEER (Ω · cm²), n = 6 | 1050.34 | 1011 |
| Average LY Papp (cm/s) | 0.83 | 0.10 |
| Passive diffusion, Papp | | |
| (cm/s, 10⁻⁶, n = 3) | | |
| Atenolol A->B | 0.19 | 0.17 |
| Propranolol A->B | 17.89 | 16.05 |
| Pgp transport, Papp (cm/s, 10⁻⁶, n = 3) | | |
| Digoxin A->B | 0.07 | 0.04 |
| Digoxin B->A | 13.09 | 10.92 |
| Efflux ratio | 190.35 | 298.60 |

### Example 8

### Neither Caco-2, MDCK, nor MDR-MDCK Cell Monolayer Integrity is not Disrupted by Air Transport with Agarose-Medium in Only Basolateral Chamber of a Transwell® Tissue Culture Plate

Caco-2, MDCK, and MDR-MDCK cells were used to determine whether cells having 0.75% agarose media in only the basolateral chamber of a Transwell® plate could be shipped by air transit. The cells were seeded onto Transwell® inserts, as described previously. Prior to shipping the cells, growth media was removed from the apical and basolateral chambers of the Transwell® plate and 1.5 mL of 0.75% agarose medium was placed in the basolateral chamber. The medium was allowed to solidify and the cells were packaged for shipping, as described previously. The cells were shipped with the supplies necessary for continuing culture and performing a quality control assay on the cells. These supplies included 200 mL of DMEM media, two culture plates, 500mL of Hanks Balanced Salt Solution (HBSS), 25 mL of quality control solution containing 500 μM lucifer yellow, 10 μM atenolol, 10 μM propanolol, 10 μM digoxin, and 10 μM pindolol or 5 μM estrone-3-sulfate, 2 mL of 10 μM lucifer yellow included for standard curve preparation, one pre-labeled 96-well dyno block, and lid to fit the box for sample collection. All of the cells were then shipped via FedEx overnight to Jefferson City, Tenn. 37760.

Detailed instructions were provided to the recipient regarding how to properly handle the cells. In brief, upon receiving the cells warmed DMEM media was placed in the basolateral (1 mL) and apical chamber (0.5 mL) of each tissue culture insert—without removing the agar-supplemented medium. After a two hour incubation at under normal growth conditions, the tissue culture inserts were moved to a new plate, provided in the shipment, containing new media in the wells. MDCK and MDR1-MDCK cells needed to incubate at least overnight before the integrity of the cell monolayer was assessed, while Caco-2 cells needed to incubate anywhere from 3-5 days before use. Feeding instructions were also provided for both cell lines.

After the cell lines were incubated for the appropriate number of days, experiments were performed to assess monolayer integrity: TEER readings were obtained, bidirectional transport assays were preformed using the provided quality control solutions, and monolayer permeability was assessed using lucifer yellow. All collected samples were places into a provided 96-well dyno block, frozen and shipped back to the inventors for LC/MS analysis. The results of the LC/MS analysis are provided in tables 11-13. The results of the experiments indicate that the aforementioned shipping and receiving procedures have no detrimental effects on the monolayer integrity of the cells tested, namely, NDCK, MDR-MDCK, Caco-2 and C2BBe1 cells. After an appropriate recovery period, the shipped cell monolayers are suitable for transport experiments.

TABLE 11
Summary of QC results for control and shipped Caco-2 cells

| | Shipped | Control |
|---|---|---|
| cell type | Caco-2 | Caco-2 |
| Seed date | Nov. 29, 2008 | Nov. 29, 2008 |
| Shipped date | Dec. 15, 2008 | N/A |
| Date of Assay | Dec. 22, 2008 | Dec. 19, 2008 |
| passage # | 64 | 64 |
| Day in culture | 23 D | 20 D |
| TEER (ohm-cm2) | 591 | 462 |
| LY Papp | 0.11 | 0.17 |
| Passive diffusion, Papp (×10⁻⁶, cm/s) | | |
| Atenolol A->B | 0.32 | 0.33 |
| Pindolol A->B | 14.19 | 13.90 |
| Propanolol A->B | 14.71 | 20.70 |
| Pgp transport, Papp (×10⁻⁶, cm/s) | | |
| Digoxin A->B | 1.13 | 0.92 |
| Digoxin B->A | 15.22 | 9.98 |
| Efflux ratio | 13.44 | 10.85 |

TABLE 12
Summary of QC results for control and shipped MDCK cells

| | Shipped | Control |
|---|---|---|
| cell type | MDCK | MDCK |
| Seed date | Dec. 1, 2008 | Dec. 1, 2008 |
| Shipped date | Dec. 8, 2008 | N/A |
| Date of Assay | Dec. 10, 2008 | Dec. 8, 2008 |
| passage # | 39 | 39 |
| Day in culture | 9 d | 7 d |
| TEER (ohm-cm2) | 3736 | 2885 |
| LY Papp A->B | 0.31 | 0.13 |
| Passive diffusion, Papp (×10⁻⁶, cm/s) | | |
| Atenolol A->B | 0.04 | 0.07 |
| Pindolol A->B | 2.57 | 5.18 |
| Pgp transport, Papp (×10⁻⁶, cm/s) | | |
| Digoxin A->B | 0.16 | 0.12 |
| Digoxin B->A | 7.44 | 6.92 |
| Efflux ratio | 46.50 | 56.00 |

TABLE 13
Summary of QC results for control and shipped MDR1-MDCK cells

| | Shipped | Control |
|---|---|---|
| Cell type | MDR1-MDCK | MDR1-MDCK |
| Seed date | Feb. 2, 2009 | Feb. 2, 2009 |
| Shipped date | Feb. 10, 2009 | N/A |
| Date of Assay | Feb. 12, 2008 | Feb. 9, 2008 |
| passage # | 19 | 19 |
| Day in culture | 10 d | 7 d |
| LY Papp A->B | 0.23 | 0.05 |
| Passive diffusion, Papp (×10⁻⁶, cm/s) | | |
| Atenolol A->B | 0.11 | 0.03 |
| Propranolol A->B | 10.52 | 15.96 |
| Pgp transport, Papp (×10⁻⁶, cm/s) | | |
| Digoxin A->B | 0.09 | 0.02 |
| Digoxin B->A | 20.11 | 12.62 |
| Efflux ratio | 220.94 | 608.03 |

### Example 9

### Altering Seed Density and Sodium Butyrate Addition Facilitate Recovery of Cell Monolayers Exposed to Simulated Transport Conditions

Experiments were conducted to determine if the pre- and post-transport incubation periods described above could be shortened without compromising cell monolayer integrity following transport. To investigate this possibility, a 7-day cell culture procedure was devised that made use of increased cell seed density and the addition of sodium butyrate to the cell culture medium.

Caco-2 cells were used to assess whether cells grown using the 7-day culture procure were likely to yield intact cell monolayers following shipment. To shorten the period required for cell monolayer formation the seeding density for each Transwell® insert was increased to 240,000 cells per cm² (rather than 60,000 cells/cm²). Once plated, the cells were incubated overnight at 37° C. with 0.5 mL of medium (90% Dulbecco's Modified Eagle Medium supplemented with 10% fetal bovine serum) in the apical chamber and 1.5 mL of medium in the basolateral chamber to allow for attachment to the Transwell®. Adherent cells were provided with fresh cell culture medium for the next two days. The cells were then left on a laboratory bench, at room temperature, overnight to simulate transport conditions. In order to simulate receipt of transported cells, the next day the Transwell® inserts were removed from tissue culture plates containing 0.75% agarose medium and moved to a new plate containing fresh tissue culture medium. The cell monolayers were then incubated for 3 or 6 days under normal growth conditions before they were tested for membrane integrity and permeability. The results of the integrity and permeability studies suggested that these cell monolayers were not acceptable for use as epithelia model system (data not shown).

Studies were then conducted to determine whether the 7-day culture procure could produce acceptable cell monolayers, following simulated transport, if cell monolayers seeded at high density were incubated in cell culture medium supplemented with sodium butyrate. Experiments were initially conducted to determine effective dosing amounts and whether sodium butyrate would adversely affect the integrity or permeability of Caco-2 cell monolayers. The experimental results suggested that cell monolayers responded best when the cell culture medium was supplemented with 4 mM sodium butyrate (data not shown).

To determine the effect of incubating cell monolayers in sodium butyrate-supplemented medium prior to transport, the tissue culture medium of some cell monolayers was supplemented with 4 mM sodium butyrate for three days prior to simulated transport. Studies were also conducted to determine the affect of incubating cell monolayers in sodium butyrate-supplemented medium following transport, but not prior to transport. Accordingly, the tissue culture medium of some cell monolayers was supplemented with 4 mM sodium butyrate following simulated transport. Cells were prepared for transport by removing the growth media from the apical and basolateral chambers of the Transwell® and adding 1.0 mL of 0.75% agarose medium to the basolateral chamber only. The medium was allowed to solidify and the tissue culture plates were wrapped with parafilm. The cells were then left on a laboratory bench, at room temperature, overnight to simulate transport conditions. In order to simulate receipt of transported cells, the next day the Transwell® inserts were removed from tissue culture plates containing 0.75% agarose medium and moved to a new plate where tissue culture medium either supplemented with, or lacking, 4 mM sodium butyrate was added to the apical and basolateral chambers of the Transwell®. The cell monolayers were incubated at 37° C. for the next three days.

The integrity and permeability of the cell monolayers was assessed in comparison to the control Caco-2 cells that were maintained under normal growth conditions (Table 15). The results indicate that simulated transport is not detrimental to cell monolayer integrity when cell monolayers are shipped with agarose medium located only on the basolateral side of the Transwell . These data also indicate that the cell monolayers remain intact and can function for up to at least two weeks after delivery. Furthermore, the data in Table 14 suggests that the addition of 4 mM sodium butyrate to cell monolayers for an additional three days prior to transport, but not flowing transport, adversely affects monolayer integrity and permeability following transport. Conversely, the data in Table 15 suggest that the addition of sodium butyrate to tissue culture medium following transport, but not prior to transport, does not disrupt cell monolayer integrity or permeability.

TABLE 14
Pre-transport addition of 4 mM sodium butyrate to Caco-2 cell monolayers
produced using a 7-day cell culture procedure causes monolayer disruption following
simulated transport.
Cell type ASI-4
Seeding Density (cells/cm²) 240,000

| | Simulated Shipped | | | Control | | |
|---|---|---|---|---|---|---|
| Passage # | 68 | | | | | |
| Seed date | May 26, 2009 | | | | | |
| Days in culture when shipped (day) | 6 | | | N/A | | |
| Days in culture when assayed (day) | 8 | 10 | 14 | 7 | 10 | 14 |
| TEER (ohm-cm²), n = 6 | 488 | 809 | 933 | 418 | 885 | 1090 |
| LY Papp (×10⁻⁶, cm/s), n = 3 | 0.19 | 0.79 | 0.65 | 0.23 | 0.18 | 0.32 |
| Passive diffusion, Papp (×10⁻⁶, cm/s), n = 3 | | | | | | |
| Atenolol A->B | 0.43 | 0.80 | 1.01 | 0.38 | 0.28 | 0.53 |
| Propanolol A->B | 15.74 | 14.46 | 17.13 | 15.94 | 10.67 | 13.62 |
| Pgp transport, Papp (×10⁻⁶, cm/s), n = 3 | | | | | | |
| Digoxin A->B | 1.19 | 2.10 | 1.88 | 1.25 | 0.93 | 1.15 |
| Digoxin B->A | 21.92 | 21.02 | 18.21 | 19.30 | 21.02 | 27.20 |
| Efflux ratio | 18.35 | 10.02 | 9.70 | 15.49 | 22.62 | 23.61 |
| BCRP transport, Papp (×10⁻⁶, cm/s) | | | | | | |
| E3S A->B | 0.62 | 0.89 | 0.72 | 0.59 | 0.31 | 0.49 |
| E3S B->A | 21.84 | 27.04 | 17.66 | 23.72 | 31.38 | 31.06 |
| Efflux ratio | 35.32 | 30.49 | 24.37 | 40.33 | 102.73 | 63.32 |

TABLE 15
(batches 1-3) The integrity of Caco-2 cell monolayers produced using a 7-day cell
culture procedure is not disrupted by simulated transport.

| | Simulated | |
|---|---|---|
| Batch 1 | shipped | Control |
| Passage # | 68 | |
| Seed date | May 14, 2009 | |
| Days in culture when shipped (day) | 3 | N/A |

| | | | | |
|---|---|---|---|---|
| Days in culture when assayed (day) | 8 | 12 | 8 | 12 |
| TEER (ohm-cm²), n = 6 | 676 | 537 | 524 | 492 |
| LY Papp (×10⁻⁶, cm/s), n = 3 | 0.18 | 0.28 | 0.18 | 0.17 |
| Passive diffusion, Papp (×10⁻⁶, cm/s), n = 3 | | | | |
| Atenolol A->B | 0.23 | 0.36 | 0.19 | 0.18 |
| Propanolol A->B | 15.91 | 15.13 | 14.30 | 15.72 |
| Pgp transport, Papp (×10⁻⁶, cm/s), n = 3 | | | | |
| Digoxin A->B | 0.74 | 0.86 | 0.51 | 0.49 |
| Digoxin B->A | 15.90 | 20.67 | 16.00 | 22.10 |
| Efflux ratio | 21.47 | 24.06 | 31.63 | 45.41 |
| BCRP transport, Papp (×10⁻⁶, cm/s) | | | | |
| E3S A->B | 0.22 | 0.53 | 0.32 | 0.33 |
| E3S B->A | 12.67 | 24.46 | 13.52 | 25.52 |
| Efflux ratio | 57.35 | 46.55 | 41.79 | 76.32 |

| Batch 2 | Simulated shipped | | | | Control | | | |
|---|---|---|---|---|---|---|---|---|
| Passage # | 64 | | | | | | | |
| Seed date | Jun. 29, 2009 | | | | Jun. 30, 2009 | | | |
| Days in culture when shipped (day) | 3 | | | | N/A | | | |
| Days in culture when assayed (day) | 8 | 11 | 15 | 17 | 7 | 10 | 14 | 16 |
| TEER (ohm-cm²), n = 6 | 448 | 593 | 769 | 920 | 503 | 563 | 659 | 897 |
| LY Papp (×10⁻⁶, cm/s), n = 3 | 0.22 | 0.27 | 0.25 | 0.16 | 0.15 | 0.20 | 0.17 | 0.16 |
| Passive diffusion, Papp (×10⁻⁶, cm/s), n = 3 | | | | | | | | |
| Atenolol A->B | 0.36 | 0.33 | 0.41 | 0.18 | 0.22 | 0.33 | 0.42 | 0.18 |
| Propanolol A->B | 17.21 | 13.28 | 22.89 | 14.81 | 14.37 | 11.89 | 23.05 | 14.18 |
| Pgp transport, Papp (×10⁻⁶, cm/s), n = 3 | | | | | | | | |
| Digoxin A->B | 1.22 | 0.90 | 1.52 | 0.73 | 0.66 | 0.80 | 1.15 | 0.57 |
| Digoxin B->A | 20.53 | 20.53 | 19.52 | 17.80 | 19.44 | 24.40 | 20.81 | 11.53 |
| Efflux ratio | 16.86 | 22.72 | 12.81 | 24.36 | 29.66 | 30.39 | 18.05 | 20.24 |
| BCRP transport, Papp (×10⁻⁶, cm/s) | | | | | | | | |
| E3S A->B | 0.43 | 0.23 | 0.46 | 0.21 | 0.33 | 0.13 | 0.40 | 0.20 |
| E3S B->A | 23.64 | 36.22 | 26.88 | 29.29 | 25.52 | 40.23 | 30.24 | 26.55 |
| Efflux ratio | 55.51 | 154.92 | 58.84 | 137.45 | 77.39 | 311.30 | 76.52 | 135.51 |

| | | |
|---|---|---|
| Batch 3 | Simulated Shipped | Control |

| | |
|---|---|
| Passage # | 66 |

| Seed date | Jul. 14, 2009 | | | Jul. 13, 2009 | | |
|---|---|---|---|---|---|---|
| Days in culture when shipped (day) | 3 | | | N/A | | |
| Days in culture when assayed (day) | 8 | 15 | 18 | 7 | 14 | 17 |
| TEER (ohm-cm²), n = 6 | 461 | 741 | 752 | 390 | 808 | 688 |
| LY Papp (×10⁻⁶, cm/s), n = 3 | 0.17 | 0.15 | 0.15 | 0.21 | 0.15 | 0.21 |
| Passive diffusion, Papp (×10⁻⁶, cm/s), n = 3 | | | | | | |
| Atenolol A->B | 0.28 | 0.15 | 0.15 | 0.33 | 0.13 | 0.22 |
| Propanolol A->B | 28.52 | 13.32 | 21.29 | 21.63 | 11.74 | 17.12 |
| Pgp transport, Papp (×10⁻⁶, cm/s), n = 3 | | | | | | |
| Digoxin A->B | 1.34 | 0.45 | 0.43 | 1.18 | 0.32 | 0.29 |
| Digoxin B->A | 21.65 | 15.98 | 18.03 | 20.85 | 20.26 | 16.88 |
| Efflux ratio | 16.14 | 0.33 | 41.55 | 17.72 | 64.14 | 57.70 |
| BCRP transport, Papp (×10⁻⁶, cm/s) | | | | | | |
| E3S A->B | 0.34 | 0.14 | 0.24 | 0.41 | 0.12 | 0.21 |
| E3S B->A | 23.48 | 28.11 | 31.26 | 22.62 | 26.22 | 29.29 |
| Efflux ratio | 68.85 | 193.95 | 127.87 | 55.09 | 220.84 | 137.05 |

What is claimed:

1. A cell culture comprising a tissue culture plate having a permeable tissue culture plate insert therein, said permeable tissue culture plate insert providing the tissue culture plate with an apical chamber and a basolateral chamber, said permeable tissue culture insert having cells deposited thereon, said apical chamber being essentially free of tissue culture medium and said basolateral chamber containing a solidifiable form of tissue culture medium.

2. The cell culture of claim 1, said cell culture comprising cells that are viable after at least 5 hours in an ambient environment.

3. The cell culture of claim 2, wherein the transepithelial electrical resistance of the cell culture is at least 300 Ω·cm².

4. The cell culture of claim 2, wherein the lucifer yellow permeability coefficient of the cell culture is less than 0.4×10⁻⁶ cm/s 48 hours after transport.

5. The cell culture of claim 1, said cell culture comprising cells that are mammalian cells.

6. The cell culture of claim 1, said cell culture comprising cells that are epithelial cells.

7. The cell culture of claim 6, said cell culture comprising cells that are mammalian epithelial cells.

8. The cell culture of claim 7, wherein the cells are MDCK cells, MDR-MDCK cells, Caco-2 cells, CEBBe1 cells, HT-29 cells, T-84 cells, or genetically engineered cells derived therefrom.

9. The cell culture of claim 8, wherein the genetically engineered cells are a Caco-2 cell expressing a form of RNA that reduces the expression of BCRP, a Caco-2 cell expressing a form of RNA that reduces the expression of PGP, a Caco-2 cell expressing a form of RNA that reduces the expression of MRP2, or an MDCK cell expressing MDR1.

10. The cell culture of claim 1, said cell culture comprising cells that are polarized.

11. The cell culture of claim 10, said cell culture comprising cells that are in the form of a polarized cell monolayer.

12. The cell culture of claim 11, wherein the transepithelial electrical resistance of the polarized cell monolayer is at least 300 Ω-cm².

13. The cell culture of claim 11, wherein the lucifer yellow permeability coefficient of the polarized cell monolayer is less than 0.4×10⁻⁶ cm/s 48 hours after transport.

14. The cell culture of claim 1, wherein the solidifiable form of tissue culture medium comprises a tissue culture medium having a solidifying agent that is gelatin, collagen, or agarose or mixtures thereof.

15. The cell culture of claim 14, wherein the solidifying agent is agarose.

16. The cell culture of claim 15, wherein the agarose comprises at least about 0.3% (weight to volume) of the solidifiable medium.

17. A method of transporting cells, said method comprising transporting a cell culture comprising a tissue culture plate having a permeable tissue culture plate insert therein, said permeable tissue culture plate insert providing the tissue culture plate with an apical chamber and a basolateral chamber, said permeable tissue culture insert having cells deposited thereon, said apical chamber being essentially free of tissue culture medium and said basolateral chamber containing a solidifiable form of tissue culture medium.

18. The cell culture of claim 17, said cell culture comprising cells that are mammalian cells.

19. The cell culture of claim 17, said cell culture comprising cells that are epithelial cells.

20. The cell culture of claim 19, said cell culture comprising cells that are mammalian epithelial cells.

21. The cell culture of claim 20, wherein the cells are MDCK cells, MDR-MDCK cells, Caco-2 cells, CEBBe1 cells, HT-29 cells, T-84 cells, or genetically engineered cells derived therefrom.

22. The cell culture of claim 21, wherein the genetically engineered cells are a Caco-2 cell expressing a form of RNA that reduces the expression of BCRP, a Caco-2 cell expressing a form of RNA that reduces the expression of PGP, a Caco-2 cell expressing a form of RNA that reduces the expression of MRP2, or an MDCK cell expressing MDR1.

23. The cell culture of claim 17, said cell culture comprising cells that are polarized.

24. The cell culture of claim 23, said cell culture comprising cells that are in the form of a polarized cell monolayer.

25. The cell culture of claim 24, wherein the transepithelial electrical resistance of the polarized cell monolayer is at least 300 Ω-cm².

26. The method of claim 24, wherein the lucifer yellow permeability coefficient of the cell monolayer is less than 0.4×10⁻⁶ cm/s 48 hours after transport.

27. The method of claim 17, said method comprising transporting said cell culture for at least two hours.

28. A method of receiving cells, said method comprising receiving a cell culture comprising a tissue culture plate having a permeable tissue culture plate insert therein, said permeable tissue culture plate insert providing the tissue culture plate with an apical chamber and a basolateral chamber, said permeable tissue culture insert having cells deposited thereon, said apical chamber being essentially free of tissue culture medium and said basolateral chamber containing a solidifiable form of tissue culture medium.

29. A method of feeding a cell culture, said cell culture comprising a tissue culture plate having a permeable tissue culture plate insert therein, said permeable tissue culture plate insert providing the tissue culture plate with an apical chamber and a basolateral chamber, said permeable tissue culture plate insert having cells deposited thereon, wherein said apical chamber is essentially free of tissue culture medium and said basolateral chamber contains solidifiable tissue culture medium, said method comprising adding liquid tissue culture medium to said apical chamber and replacing said solidifiable tissue culture medium with liquid tissue culture medium.

30. The method of claim 29, wherein replacing the solidifiable tissue culture medium comprises removing the permeable tissue culture plate insert from the tissue culture plate having the solidifiable basolateral medium and placing the permeable tissue culture plate insert into a new tissue culture plate having liquid tissue culture medium.

31. A kit for transporting cells comprising a tissue culture plate having a permeable tissue culture plate insert therein, said permeable tissue culture plate insert providing the tissue culture plate with an apical chamber and a basolateral chamber, said permeable tissue culture insert having cells deposited thereon, said apical chamber being essentially free of tissue culture medium and said basolateral chamber containing a solidifiable form of tissue culture medium and optionally having instructions for transporting or handling said cells, said kit having liquid tissue culture medium.

32. The method of claim 29, wherein the liquid tissue culture medium comprises a salt of butyric acid.

33. The method of claim 29, wherein the liquid tissue culture medium comprises sodium butyrate.

34. The method of claim 33, wherein the concentration of sodium butyrate in the liquid tissue culture medium is about 4 mM.

35. The kit of claim 31, further comprising a salt of butyric acid.
